SEQUENCE LISTING (63 sequences)

SEQ ID NO: 1 - DNA, 27 bp, Artificial
  Primer
gctgcacgctacccaccaggccccctg

SEQ ID NO: 2 - DNA, 45 bp, Artificial
  Primer
ttgcggccgctcttctactagcggggcacagcacaaagctcatag

SEQ ID NO: 3 - DNA, 50 bp, Artificial
  Primer
tattctcactggccattacggccgctgcacgctacccaccaggccccctg

SEQ ID NO: 4 - DNA, 80 bp, Artificial
  Primer
tattctcactggccattacggccgtggacctgatgcaccggcgccaacgctgggctgcacgctacccaccaggccccctg

SEQ ID NO: 5 - DNA, 107 bp, Artificial
  Primer
tattctcactggccattacggccatggctctagaagcactggtgcccctggccgtgatagtggccatcttcctgctcctggtggacctgatgcaccggcgccaacgc

SEQ ID NO: 6 - DNA, 27 bp, Artificial
  Primer
gcggggcacagcacaaagctcataggg

SEQ ID NO: 7 - DNA, 28 bp, Artificial
  Primer
ctccctcctggccccactcctctcccaa

SEQ ID NO: 8 - DNA, 46 bp, Artificial
  Primer
tttgcggccgctcttctatcagacaggaatgaagcacagcctggta

SEQ ID NO: 9 - DNA, 27 bp, Artificial
  Primer
cttggaattccagggcccacacctctg

SEQ ID NO: 10 - DNA, 48 bp, Artificial
  Primer
tttgcggccgctcttctatcaggctccacttacggtgccatcccttga

SEQ ID NO: 11 - DNA, 83 bp, Artificial
  Primer
tattctcactggccattacggcctatggaacccattcacatggactttttaagaagcttggaattccagggcccacacctctg

SEQ ID NO: 12 - DNA, 50 bp, Artificial
  Primer
tattctcactggccattacggcccttggaattccagggcccacacctctg

SEQ ID NO: 13 - DNA, 87 bp, Artificial
  Primer
tattctcactggccattacggcccctcctggctgtcagcctggtgctcctctatctatatggaacccattcacatggactttttagg

SEQ ID NO: 14 - DNA, 28 bp, Artificial
  Primer
ggctccacttacggtgccatcccttgac

SEQ ID NO: 15 - DNA, 75 bp, Artificial
  Primer
tattctcactggccattacggccagacagagctctgggagaggaaaactccctcctggccccactcctctcccag

SEQ ID NO: 16 - DNA, 51 bp, Artificial
  Primer
tattctcactggccattacggccctccctcctggccccactcctctcccag

SEQ ID NO: 17 - DNA, 29 bp, Artificial
  Primer
gacaggaatgaagcacagctggtagaagg

SEQ ID NO: 18 - DNA, 57 bp, Artificial
  Primer
ctctcatgtttgcttctcctttcactctggagacagcgctctgggagaggaaaactc

SEQ ID NO: 19 - DNA, 54 bp, Artificial
  Primer
acagagcacaaggaccacaagagaatcggccgtaagtgccatagttaatttctc

SEQ ID NO: 20 - DNA, 33 bp, Artificial
  Primer
ggatcgacatatgggagactcccacgtggacac

SEQ ID NO: 21 - DNA, 34 bp, Artificial
  Primer
ccgataagcttatcagctccacacgtccagggag

SEQ ID NO: 22 - DNA, 23 bp, Artificial
  Primer
tgtgttcaagaggaagcccgctg

SEQ ID NO: 23 - DNA, 25 bp, Artificial
  Primer
gtcctcaatgctgctcttccccatc

SEQ ID NO: 24 - DNA, 24 bp, Artificial
  Primer
cttgaccttctccccaccagcctg

SEQ ID NO: 25 - DNA, 24 bp, Artificial
  Primer
gtatctctggacctcgtgcaccac

SEQ ID NO: 26 - DNA, 23 bp, Artificial
  Primer
ctgaccttctccccaccagcctg

SEQ ID NO: 27 - DNA, 22 bp, Artificial
  Primer
tgtatctctggacctcgtgcac

SEQ ID NO: 28 - DNA, 20 bp, Artificial
  Primer
gcttctccccaccagcctgc

SEQ ID NO: 29 - DNA, 24 bp, Artificial
  Primer
tcaatgtatctctggacctcgtgc

SEQ ID NO: 30 - DNA, 23 bp, Artificial
  Primer
gcattgaccttctccccaccagc

SEQ ID NO: 31 - DNA, 22 bp, Artificial
  Primer
caccacgtgctccaggtctcta

SEQ ID NO: 32 - DNA, 81 bp, Artificial
  Primer
tattctcactggccattacggccgtggacctgatgcaccggcgccaacgctgggctgcacgctactcaccaggccccctgc

SEQ ID NO: 33 - DNA, 52 bp, Artificial
  Primer
gcggggcacagcacaaagctcatagggggatgggctcaccaggaaagcaaag

SEQ ID NO: 34 - DNA, 21 bp, Artificial
  Primer
tccagatcctgggtttcgggc

SEQ ID NO: 35 - DNA, 22 bp, Artificial
  Primer
tgatgggcacaggcgggcggtc

SEQ ID NO: 36 - DNA, 21 bp, Artificial
  Primer
gccaaggggaaccctgagagc

SEQ ID NO: 37 - DNA, 21 bp, Artificial
  Primer
ctccatctctgccaggaaggc

SEQ ID NO: 38 - DNA, 24 bp, Artificial
  Primer
ccaataacagtctttccattcctc

SEQ ID NO: 39 - DNA, 24 bp, Artificial
  Primer
gagaaagaatggatccaaaaaatc

SEQ ID NO: 40 - DNA, 23 bp, Artificial
  Primer
cgaggtttgctctcatgaccatg

SEQ ID NO: 41 - DNA, 24 bp, Artificial
  Primer
tgccaatgcagtttctgggtccac

SEQ ID NO: 42 - DNA, 22 bp, Artificial
  Primer
gtctctatagctgaggatgaag

SEQ ID NO: 43 - DNA, 23 bp, Artificial
  Primer
ggcacttttcataaatcccactg

SEQ ID NO: 44 - DNA, 23 bp, Artificial
  Primer
gattctttctctcaataacagtc

SEQ ID NO: 45 - DNA, 23 bp, Artificial
  Primer
gatccaaaaaatcaaatcttaaa

SEQ ID NO: 46 - DNA, 21 bp, Artificial
  Primer
aggaagcagagacaggcaagc

SEQ ID NO: 47 - DNA, 22 bp, Artificial
  Primer
gcctcagatttctcaccaacac

SEQ ID NO: 48 - DNA, 5024 bp, Artificial
  Plasmid
ctcgagaaatcataaaaaatttatttgctttgtgagcggataacaattataatagattcaattgtgagcggataacaatttcacacagaattcattaaagaggagaaattaactatggcacttagtgggatccgcatgcgagctcggtaccccgggggtggcagcggttctggcgcagcagcggaaatcagtggtcacatcgtacgttccccgatggttggtactttctaccgcaccccaagcccggacgcaaaagcgttcatcgaagtgggtcagaaagtcaacgtgggcgataccctgtgcatcgttgaagccatgaaaatgatgaaccagatcgaagcggacaaatccggtaccgtgaaagcaattctggtcgaaagtggacaaccggtagaatttgacgagccgctggtcgtcatcgagggtggcagcggttctggccaccatcaccatcaccataagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 49 - DNA, 51 bp, Artificial
  Cloning site
atggcacttagtgggatccgcatgcgagctcggtaccccgggggtggcagc

SEQ ID NO: 50 - DNA, 51 bp, Artificial
  Cloning site
gctgccacccccggggtaccgagctcgcatgcggatcccactaagtgccat

SEQ ID NO: 51 - DNA, 4700 bp, Artificial
  Plasmid
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcccaggacccaacgctgcccgaaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcacaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacacatatgaacgactttcatcgcgatacgtgggcggaagtggatttggacgccatttacgacaatgtggcgaatttgcgccgtttgctgccggacgacacgcacattatggcggtcgtgaaggcgaacgcctatggacatggggatgtgcaggtggcaaggacagcgctcgaagcgggggcctcccgcctggcggttgcctttttggatgaggcgctcgctttaagggaaaaaggaatcgaagcgccgattctagttctcggggcttcccgtccagctgatgcggcgctggccgcccagcagcgcattgccctgaccgtgttccgctccgactggttggaagaagcgtccgccctttacagcggccctattcctattcatttccatttgaaaatggacaccggcatgggacggcttggagtgaaagacgaggaggagacgaaacgaatcgcagcgctgattgagcgccatccgcattttgtgcttgaaggggcgtacacgcattttgcgactgcggatgaggtgaacaccgattatttttcctatcagtatacccgttttttgcacatgctcgaatggctgccgtcgcgcccgccgctcgtccattgcgccaacagcgcagcgtcgctccgtttccctgaccggacgttcaatatggtccgcttcggcattgccatgtatgggcttgccccgtcgcccggcatcaagccgctgctgccgtatccattaaaagaagcattttcgctccatagccgcctcgtacacgtcaaaaaactgcaaccaggcgaaaaggtgagctatggtgcgacgtacactgcgcagacggaggagtggatcgggacgattccgatcggctatgcggacggctggctccgccgcctgcagcactttcatgtccttgttgacggacaaaaggcgccgattgtcggccgcatttgcatggaccagtgcatgatccgcctgcctgggccgctgccggtcggcacgaaggtgacactgattggtcgccagggggacgaggtaatttccattgatgatgtcgctcgccatttggaaacgatcaactacgaagtgccttgcacgatcagctatcgagtgccccgtatttttttccgccataagcgtataatggaagtgagaaacgccattggccgcggggaaagcagtgcacatcaccatcaccatcactaaaagcttggatccgaattcagcccgcctaatgagcgggcttttttttgaacaaaattagcttggctgttttggcggatgagagaaga

SEQ ID NO: 52 - DNA, 1512 bp, Homo sapiens
atggctctcatcccagacttggccatggaaacctggcttctcctggctgtcagcctggtgctcctctatctatatggaacccattcacatggactttttaagaagcttggaattccagggcccacacctctgccttttttgggaaatattttgtcctaccataagggcttttgtatgtttgacatggaatgtcataaaaagtatggaaaagtgtggggcttttatgatggtcaacagcctgtgctggctatcacagatcctgacatgatcaaaacagtgctagtgaaagaatgttattctgtcttcacaaaccggaggccttttggtccagtgggatttatgaaaagtgccatctctatagctgaggatgaagaatggaagagattacgatcattgctgtctccaaccttcaccagtggaaaactcaaggagatggtccctatcattgcccagtatggagatgtgttggtgagaaatctgaggcgggaagcagagacaggcaagcctgtcaccttgaaagacgtctttggggcctacagcatggatgtgatcactagcacatcatttggagtgaacatcgactctctcaacaatccacaagacccctttgtggaaaacaccaagaagcttttaagatttgattttttggatccattctttctctcaataacagtctttccattcctcatcccaattcttgaagtattaaatatctgtgtgtttccaagagaagttacaaattttttaagaaaatctgtaaaaaggatgaaagaaagtcgcctcgaagatacacaaaagcaccgagtggatttccttcagctgatgattgactctcagaattcaaaagaaactgagtcccacaaagctctgtccgatctggagctcgtggcccaatcaattatctttatttttgctggctatgaaaccacgagcagtgttctctccttcattatgtatgaactggccactcaccctgatgtccagcagaaactgcaggaggaaattgatgcagttttacccaataaggcaccacccacctatgatactgtgctacagatggagtatcttgacatggtggtgaatgaaacgctcagattattcccaattgctatgagacttgagagggtctgcaaaaaagatgttgagatcaatgggatgttcattcccaaaggggtggtggtgatgattccaagctatgctcttcaccgtgacccaaagtactggacagagcctgagaagttcctccctgaaagattcagcaagaagaacaaggacaacatagatccttacatatacacaccctttggaagtggacccagaaactgcattggcatgaggtttgctctcatgaacatgaaacttgctctaatcagagtccttcagaacttctccttcaaaccttgtaaagaaacacagatccccctgaaattaagcttaggaggacttcttcaaccagaaaaacccgttgttctaaaggttgagtcaagggatggcaccgtaagtggagcctga

SEQ ID NO: 53 - protein, 503 aa, Homo sapiens
MALIPDLAMETWLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 54 - DNA, 1835 bp, Homo sapiens
atggattctcttgtggtccttgtgctctgtctctcatgtttgcttctcctttcactctggagacagagctctgggagaggaaaactccctcctggccccactcctctcccagtgattggaaatatcctacagataggtattaaggacatcagcaaatccttaaccaatctctcaaaggtctatggcccggtgttcactctgtattttggcctgaaacccatagtggtgctgcatggatatgaagcagtgaaggaagccctgattgatcttggagaggagttttctggaagaggcattttcccactggctgaaagagctaacagaggatttggaattgttttcagcaatggaaagaaatggaaggagatccggcgtttctccctcatgacgctgcggaattttgggatggggaagaggagcattgaggaccgtgttcaagaggaagcccgctgccttgtggaggagttgagaaaaaccaaggcctcaccctgtgatcccactttcatcctgggctgtgctccctgcaatgtgatctgctccattattttccataaacgttttgattataaagatcagcaatttcttaacttaatggaaaagttgaatgaaaacatcaagattttgagcagcccctggatccagatctgcaataatttttctcctatcattgattacttcccgggaactcacaacaaattacttaaaaacgttgcttttatgaaaagttatattttggaaaaagtaaaagaacaccaagaatcaatggacatgaacaaccctcaggactttattgattgcttcctgatgaaaatggagaaggaaaagcacaaccaaccatctgaatttactattgaaagcttggaaaacactgcagttgacttgtttggagctgggacagagacgacaagcacaaccctgagatatgctctccttctcctgctgaagcacccagaggtcacagctaaagtccaggaagagattgaacgtgtgattggcagaaaccggagcccctgcatgcaagacaggagccacatgccctacacagatgctgtggtgcacgaggtccagagatacattgaccttctccccaccagcctgccccatgcagtgacctgtgacattaaattcagaaactatctcattcccaagggcacaaccatattaatttccctgacttctgtgctacatgacaacaaagaatttcccaacccagagatgtttgaccctcatcactttctggatgaaggtggcaattttaagaaaagtaaatacttcatgcctttctcagcaggaaaacggatttgtgtgggagaagccctggccggcatggagctgtttttattcctgacctccattttacagaactttaacctgaaatctctggttgacccaaagaaccttgacaccactccagttgtcaatggatttgcctctgtgccgcccttctaccagctgtgcttcattcctgtctgaagaagagcagatggcctggctgctgctgtgcagtccctgcagctctctttcctctggggcattatccatctttgcactatctgtaatgccttttctcacctgtcatctcacattttcccttccctgaagatctagtgaacattcgacctccattacggagagtttcctatgtttcactgtgcaaatatatctgctattctccatactctgtaacagttgcattgactgtcacataatgctcatacttatctaatgtagagtattaatatgttattattaaatagagaaatatgatttgtgtattataattcaaaggcatttcttttctgcatgatctaaataaaaagcattattatttgctg

SEQ ID NO: 55 - protein, 490 aa, Homo sapiens [sequence omitted]

SEQ ID NO: 56 - protein, 44 aa, Homo sapiens
RRADGLAAAVQSLQLSFLWGIIHLCTICNAFSHLSSHIFPSLKI

SEQ ID NO: 57 - protein, 24 aa, Homo sapiens
TFDLHYGEFPMFHCANISAILHTL

SEQ ID NO: 58 - protein, 10 aa, Homo sapiens
LSHNAHTYLM

SEQ ID NO: 59 - protein, 11 aa, Homo sapiens
SINMLLLNREI

SEQ ID NO: 60 - protein, 14 aa, Homo sapiens [sequence omitted]

SEQ ID NO: 61 - protein, 7 aa, Homo sapiens
IKSIIIC

SEQ ID NO: 62 - DNA, 1494 bp, Homo sapiens
atggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcacatgccctacaccactgccgtgattcatgaggtgcagcgctttggggacatcgtccccctgggtatgacccatatgacatcccgtgacatcgaagtacagggcttccgcatccctaagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctag

SEQ ID NO: 63 - protein, 497 aa, Homo sapiens
MGLEALVPLAVIVAIFLLLVDLMHRRQRWAARYPPGPLPLPGLGNLLHVDFQNTPYCFDQLRRRFGDVFSLQLAWTPVVVLNGLAAVREALVTHGEDTADRPPVPITQILGFGPRSQGVFLARYGPAWREQRRFSVSTLRNLGLGKKSLEQWVTEEAACLCAAFANHSGRPFRPNGLLDKAVSNVIASLTCGRRFEYDDPRFLRLLDLAQEGLKEESGFLREVLNAVPVLLHIPALAGKVLRFQKAFLTQLDELLTEHRMTWDPAQPPRDLTEAFLAEMEKAKGNPESSFNDENLRIVVADLFSAGMVTTSTTLAWGLLLMILHPDVQRRVQQEIDDVIGQVRRPEMGDQAHMPYTTAVIHEVQRFGDIVPLGMTHMTSRDIEVQGFRIPKGTTLITNLSSVLKDEAVWEKPFRFHPEHFLDAQGHFVKPEAFLPFSAGRRACLGEPLARMELFLFFTSLLQHFSFSVPTGQPRPSHHGVFAFLVSPSPYELCAVPR

(19) United States
(12) Patent Application Publication
(10) Pub. No.: US 2009/0239761 A1
(43) Pub. Date: Sep. 24, 2009

(54) Protein arrays and uses thereof

(76) Inventors: Jonathan Michael Blackburn, Cambridge (GB); Benjamin Leslie James Godber, Cambridge (GB); Darren James Hart, Berkshire (GB); Nicholas A. Bockett, Christchurch (NZ); Roland Kozlowski, Woodstock (GB); Michael Dyson, Cambridge (GB)

Correspondence Address: MINTZ LEVIN COHN FERRIS GLOVSKY & POPEO, ONE FINANCIAL CENTER, BOSTON, MA, 02111, US

(21) Appl. No.: 12/291,466

(22) Filed: Nov. 10, 2008

Related U.S. Application Data
(63) Continuation of application No. 10/527,603, filed on Mar. 27, 2006, now abandoned.
Application No. 10/527,603 is a continuation of application No. PCT/GB2002/005499, filed on Dec. 5, 2002.
Application No. PCT/GB2002/005499 is a continuation-in-part of application No. 60/410,815, filed on Sep. 16, 2002.
Application No. 60/410,815 is a continuation-in-part of application No. 10/313,963, filed on Dec. 5, 2002.
(60) Provisional application No. 60/410,815, filed on Sep. 16, 2002.

(51) Int. Cl. C40B 30/04; C40B 40/10; C40B 50/06; C40B 50/00; C40B 30/00; C12N 9/00

(52) U.S. Cl. 506/9; 506/18; 506/26; 506/23; 506/7; 435/183

(57) ABSTRACT

The inventors herein describe methods for the production of a functional human, animal, plant or microbe protein arrays and methods to assay for interactions between the proteins on the array with molecules of interest, for example, using such arrays to determine the in vitro metabolite profile of any drug. Such protein arrays can be used, for example, to assay, in a parallel fashion, the protein products of DNA sequences encoding drug metabolizing enzymes (DMES) to obtain a toxicology profile. Also described herein is a novel DME expression and purification strategy using detergents and not requiring an ultra-centrifugation step.

### BACKGROUND OF THE INVENTION

1. Field of the Invention

This invention relates to molecular biology and drug discovery.

2. Background of the Related Art

It is estimated that greater than 90% of drugs that enter human clinical trials fail to be approved as a drug by the regulatory authorities mainly due to a low therapeutic index (median toxic dose/median effective dose). In many cases the mechanism of toxicity of a drug candidate is unknown and without this understanding there is no assurance that a replacement drug candidate will not fail for the same reasons.

Since the advances in molecular biology and combinatorial chemistry in the late 1980s, the drug discovery process, with its emphasis on potency, has become more efficient in finding new drug leads. Unfortunately advances in drug development, with its emphasis on safety and toxicity, have not kept pace with the increases in efficiency of drug discovery, and this has become a bottleneck in the overall process of new drug approval. The most potent drug leads are taken forward to the drug development stage and become drug candidates: first undergoing preclinical toxicology studies in tissue culture cell viability assays and animal studies, prior to the commencement of human clinical trials to gain regulatory authority approval. If pre-clinical toxicology studies were more predictive of the clinical outcome this would improve the success rate of drug clinical trials dramatically. In addition, if pre-clinical toxicology and pharmacology studies could keep pace with drug discovery then the two processes could be integrated so that the toxicology profiles of new chemical entities (NCEs) could be rapidly fed back to the drug discovery team in a synergistic process to identify drug candidates with a potentially superior therapeutic index in pre-clinical and clinical trials.

Drugs are often metabolised in vivo by the drug metabolizing enzymes (DMEs) and the therapeutic index of a drug is determined in large part by its interactions with these enzymes. DMEs are normally classified as Phase 1 or Phase 2 enzymes.

Phase 1 DMEs, which include the cytochrome P450s and flavin monooxygenases (FMOs), are responsible for the initial bio-transformation of xenobiotics and drugs and catalyse the introduction of an oxygen atom into substrate molecules. Presently, more than 57 human cytochrome P450 genes have been sequenced. Amongst these, CYP3A4, CYP2D6 and the CYP2C subfamily are responsible for the primary metabolism of the majority of current drugs (for example CYP3A4 is known to metabolise more than 120 different drugs including acetorninophen, codeine, cyclosporin A, diazepam, erythromycin, lidocaine, lovastatin, taxol, and warfarin) and are found to be polymorphic within the population (for example more than 70 different alleles have been reported for CYP2D6).

Phase 2 DMEs, which include UDP-glycosyltransferases, glutathione S-transferases, sulfotransferases and N-acetyltransferases, aid in both excretion and de-toxification processes by conjugating soluble groups, such as acetyl, glucuronide, glutathione and sulphate, to both the primary drugs and the metabolites produced by the phase 1 DMEs.

There are three main mechanisms by which drugs can interact with DMEs.

1. A drug might inhibit one or more DME, or it might act as a turn-over substrate with a DME resulting in the production of metabolites and secondary metabolites with their own toxicology profiles. For example, oxidation of drugs by Phase I DMEs often leads to hydroxylated or dealkylated metabolites which, as in the case of cocaine, can act as strong electrophiles and can covalently modify DNA or proteins, thus leading to toxic effects.

2. A drug might induce expression of a specific set of DMEs by activating transcription through binding to a nuclear receptor, examples of which include: the aryl hydrocarbon receptor (AhR) which up-regulates P450 1A1 and the glutathione S transferases (GSTs); the glucocorticoid and androstane receptors which up-regulate P450 2C9; and the pregnane X receptor which up-regulates the P450 3A family.

3. A drug might modulate intracellular drug concentrations through interaction with drug transporters such as P-glycoprotein or the multi-drug resistant proteins (MDR1-5).

Each of these mechanisms can affect not only the metabolism and possible toxicity of the drug itself but can also lead to adverse drug-drug interactions by directly or indirectly affecting the metabolism of other compounds. Thus, a drug might inhibit a P450 which would otherwise detoxify a second compound (for example quinidine is metabolized by the CYP3A4 enzyme but it is a potent inhibitor of CYP2D6), or it might induce expression of a P450 which then turns-over a second compound to a toxic metabolite, or it might inhibit entry of another compound into a cell, leading to altered effects of the second compound. For example, mibefradil, a calcium T- and L-channel blocker developed for use in hyper-tension, was recently removed from the market after reports of severe drug-drug interactions. It was found that the mode of action of toxicity of mibefradil was its potent inhibition of both P450 3A4 and P-glycoprotein. It is therefore increasingly important that these potential effects are assessed for each drug candidate at as early a stage in the drug development process as possible since a large proportion of adverse drug-drug interactions should be predictable once the basic pharmacology is known.

Pre-clinical toxicology studies are usually performed by tissue culture cell viability assays and animal studies. However, immortalised cell lines may not give a true indication of the in vivo toxicity of a drug, especially regarding its interaction with DMEs due to expression level differences between immortalised cells and normal cells. Animal models can give a useful indication of toxicity, but there are several reports of drugs showing different toxic effects in humans and rodents. These differences in toxic effects can arise for a number of reasons. For example, human and rodent P450s might be inhibited by drugs to different extents, or the drugs might be oxidised with different regio-selectivities to yield distinct metabolites, or, as in the case of tamoxifen, the expression levels of phase 1 or phase 2 enzymes might vary and result in different metabolites being formed. Transgenic mice, with human nuclear receptor genes, are now being used in drug toxicology experiments, but this technology is at a very early stage and in theory one would need to clone all human drug interacting proteins and express them at the appropriate levels in order to have a complete drug, metabolite and secondary metabolite toxicology profile.

Toxicogenomics and pharmacogenomics are defined as the application of gene expression technology to toxicology and pharmacology. Here the ability of a drug to induce gene expression (through binding to nuclear receptors or other mechanisms) is assessed either in tissue culture cell lines or animal models. Gene expression can be monitored either at the RNA level (using DNA micro-arrays) or at the protein level (using 2D protein gels). Gene families are sometimes seen to be up-regulated, an example being DMEs through the drug binding to the relevant nuclear receptor or through the drug or its metabolites causing inflammation, DNA damage, oxidative stress or cell signalling. A critique of this approach is that it is an end-point assay giving information on how a cell tries to cope with the introduction of a foreign drug, but gives no information on the mechanism by which the drug exerted that effect. For example the knowledge that a drug causes the up-regulation of genes associated with DNA damage gives no information regarding which enzymes oxidised the drug in the first place to produce the resulting electrophilic intermediates capable of covalently modifying DNA. Also a comparison of the human and mouse pregnane X receptors (PXRs) revealed marked differences in their activation by certain drugs questioning the relevance of animal toxicogenomic studies for predicting a drug's effect in humans.

The problems associated with some of the current methods to determine pre-clinical toxicology detailed above strongly argues for more complete and rigorous in vitro screening of drugs against human drug interacting proteins. In order to fully test a drug for potential toxicity one would wish to assay for binding, inhibition and turn-over with the full complement, or a significant proportion of human DMEs, nuclear receptors and drug transport proteins. Currently however this would be extremely time consuming and laborious both because of the limited numbers of human drug interacting proteins cloned, expressed and purified in a functional form and because each protein would require the establishment of a unique assay to detect drug binding, inhibition or enhancement of activity, and analysis of metabolite production.

In contrast to arrays of DNA or gels of denatured proteins, arrays of active proteins could be used to provide much of this detailed mechanistic information in a high throughput, quantitative manner and would complement data obtained by conventional means. However, thus far there has been no example of a protein array incorporating DMEs, nuclear receptors, or drug transporter systems in a folded, fully functional state.

### BRIEF SUMMARY OF THE INVENTION

The Inventors herein describe methods for the production of a functional human, animal, plant or microbe protein arrays and methods to assay for interactions between the proteins on the array with molecules of interest for example, using such arrays to determine the in vitro metabolite profile of any drug. Such protein arrays can be used, for example, to assay, in a parallel fashion, the protein products of DNA sequences encoding drug metabolising enzymes (DMEs) to obtain a toxicology profile. Also described herein is a novel DME expression and purification strategy using detergents and not requiring an ultra-centrifugation step. All previously reported P450 purification approaches have required an ultracentrifugation step which means that it is difficult to perform P450 purifications in a multiplexed manner.

Drug metabolising enzymes represent a specific subset of the overall collection of proteins in a given cell, tissue or organism that can have particular clinical and pharmaceutical relevance. Protein arrays comprising this protein group represent a highly versatile tool with potential applications in drug target identification and validation processes as well as in drug selectivity and toxicity screens and in delineation of drug metabolising enzyme-protein interaction maps. However, for such applications to be viable, the drug metabolising enzymes on the array need to be correctly folded such that they are likely to retain many if not all aspects of their natural function. Such an array has not previously been described for a number of reasons. Firstly, it is entirely dependent upon the ability to generate an appropriate collection of expressed, purified and functional proteins; this is known in the art to be technically challenging. Secondly it depends on the ability to immobilise each protein onto a suitable surface such that they maintain function and it is not immediately obvious how this could be achieved for DMEs; many of these proteins, such as the P450s, are membrane-associated and additionally require accessory proteins in order to be catalytically activated in the same manner as within a cell, yet often no stable complex is formed between the DME and the accessory protein (an example here is the transient interaction between cytochrome P450s and the NADPH-cytochrome P450 reductase).

In vitro screening of protein interactions in an array format has been demonstrated in the prior art. In its simplest form, microarrays have been generated from immunoglobulin molecules in order to capture proteins from solution. These antibody arrays provide miniaturisation of the ELISA assay and enable high throughput analysis of, for example, cell lysates, serum samples or recombinant protein mixtures. A second example of protein array types is the antigen array, used to identify auto-antibodies in serum samples. In these cases, the antigens are arrayed on a denaturing surface, making all linear epitopes available for antibody binding but destroying the native form of the arrayed molecules. Two examples of protein arrays in which the proteins were arrayed to retain correct folding and function have recently been described. In the first example, a ‘proteome on a chip’ was created for the relatively small yeast genome, enabling the researchers to identify activities based on binding to individual proteins in their native conformations. In the second example, a small array of protein kinases was created and probed for function. In addition, arrays of specifically selected, functional proteins that have been precisely tagged at the N- or C-terminus have been created and interrogated to identify interacting partners such as DNA and small molecules. In each of these cases, individual proteins were purified and deposited singly onto the array. To date, there has been no description of an array of folded, drug metabolising enzymes, nor has there been a description of a protein array where two or more proteins are required to form an active complex.

Currently all in vitro, non-cell-based phase 1 and 2 drug metabolism assays have been performed in solution phase assays and in principle it would be possible to individually assay a collection of DME proteins in a test tube format. However the serial nature of this work, the large sample volumes involved, and the poor compatibility of an individual solution phase assay platform across a range of different assay types (for example, drug binding, turn-over, and cytotoxicity assays) make this approach cumbersome and unattractive and also makes accurate, comparative kinetic analysis difficult.

There is still a lack of high throughput tools for the functional study of drug metabolising enzymes and also a lack of tools to assay the effects of drug molecules on these functions in parallel. As the numbers of drug metabolising enzymes may approach the hundreds, if not the thousands, a highly parallel method of functional analysis is needed that does not require antibodies, gels or beads for it to be performed.

### BRIEF DESCRIPTION OF THE DRAWINGS

FIG. 1A shows a plasmid map of pBJW102.2 for expression of C-terminal BCCP hexa-histidine constructs.

FIG. 1B shows the DNA sequence of pBJW102.2

FIG. 1C shows the cloning site of pBJW102.2 from start codon. Human P450s, NADPH-cytochrome P450 reductase, and cytochrome b5 ORFs, and truncations thereof, were ligated to a DraIII/SmaI digested vector of pBJW102.2.

FIG. 2A shows a vector map of pJW45

FIG. 2B shows the sequence of the vector pJW45

FIG. 3A shows the DNA sequence of Human P450 3A4 open reading frame.

FIG. 3B. shows the amino acid sequence of full length human P450 3A4.

FIG. 4A shows the DNA sequence of human P450 2C9 open reading frame.

FIG. 4B shows the amino acid sequence of full length human P450 2C9

FIG. 5A shows the DNA sequence of human P450 2D6 open reading frame.

FIG. 5B shows the amino acid sequence of full length human P450 2D6.

FIG. 6 shows a western blot and coomassie-stained gel of purification of cytochrome P450 3A4 from E. coli. Samples from the purification of cytochrome P450 3A4 were run on SDS-PAGE, stained for protein using coomassie or Western blotted onto nitrocellulose membrane, probed with streptavidin-HRP conjugate and visualised using DAB stain:

Lanes 1: Whole cells

### Lanes 2: Lysate

Lanes 3: Lysed E. coli cells
Lanes 4: Supernatant from E. coli cell wash
Lanes 5: Pellet from E. coli cell wash
Lanes 6: Supernatant after membrane solubilisation
Lanes 7: pellet after membrane solubilisation
Lanes 8: molecular weight markers: 175, 83, 62, 48, 32, 25, 16.5, 6.5 Kda

FIG. 7 shows the Coomassie stained gel of Ni-NTA column purification of cytochrome P450 3A4. Samples from all stages of column purification were run on SDS-PAGE:

### Lane 1: Markers 175, 83, 62, 48, 32, 25, 16.5, 6.5 KDa

Lane 2: Supernatant from membrane solublisation

### Lane 3: Column Flow-Through

Lane 4: Wash in buffer C
Lane 5: Wash in buffer D
Lanes 6&7: Washes in buffer D+50 mM Imidazole
Lanes 8-12: Elution in buffer D+200 mM Imidazole

FIG. 8 shows the assay of activity for cytochrome P450 2D6 in a reconstitution assay using the substrate AMMC. Recombinant, tagged CYP2D6 was compared with a commercially available CYP2D6 in terms of ability to turnover AMMC after reconstitution in liposomes with NADPH-cytochrome P450 reductase.

FIG. 9 shows the rates of resorufin formation from BzRes by cumene hydrogen peroxide activated cytochrome P450 3A4. Cytochrome P450 3A4 was assayed in solution with cumene hydrogen peroxide activation in the presence of increasing concentrations of BzRes up to 160 μM.

FIG. 10 shows the equilibrium binding of [³H]ketoconazole to immobilised CYP3A4 and CYP2C9. In the case of CYP3A4 the data points are the means i standard deviation, of 4 experiments. Non-specific binding was determined in the presence of 100 μM ketoconazole (data not shown).

FIG. 11 shows the chemical activation of tagged, immobilised P450 involving conversion of DBF to fluorescein by CHP activated P450 3A4 immobilised on a streptavidin surface.

FIG. 12 shows the stability of agarose encapsulated microsomes. Microsomes containing cytochrome P450 2D6 plus NADPH-cytochrome P450 reductase and cytochrome b5 were diluted in agarose and allowed to set in 96 well plates. AMMC turnover was measured immediately and after two and seven days at 4° C.

FIG. 13 shows the turnover of BzRes by cytochrome P450 3A4 isoforms. Cytochrome P450 3A4 isoforms WT, *1, *2, *3, *4, *5 & *15, (approximately 1 μg) were incubated in the presence of BzRes (0-160 μM) and cumene hydrogen peroxide (200 μM at room temperature in 200 mM KPO₄ buffer pH 7.4. Formation of resorufin was measured over time and rates were calculated from progress curves. Curves describing conventional Michaelis-Menton kinetics were fitted to the data.

FIG. 14 shows the inhibition of cytochrome P450 3A4 isoforms by ketoconazole. Cytochrome P450 3A4 isoforms WT, *1, *2, *3, *4, *5 & *15, (approximately 1 μg) were incubated in the presence of BzRes (50 μM), Cumene hydrogen peroxide (200 μM) and ketoconazole (0, 0.008, 0.04, 0.2, 1, 5 μM) at room temperature in 200 mM KPO₄ buffer pH 7.4. Formation of resorufin was measured over time and rates were calculated from progress curves. IC₅₀ inhibition curves were fitted to the data.

### DETAILED DESCRIPTION OF THE INVENTION

In a first aspect, the invention provides a protein array comprising a surface having a plurality of spatially defined locations wherein at each location there are deposited at least two protein moieties which are capable of forming a complex characterised in that said complex is transiently formed. Such complexes are transiently (i.e. momentarily) formed, for example, during enzyme catalysis or during a binding event, such as the dimerisation of a receptor upon binding a ligand or the formation of a complex of DNA binding proteins prior to the binding of further proteins to bring about catalysis.

Each position in the pattern of an array of the first aspect contains, for example, a sample of two or more protein types wherein said two or more proteins are required to form a complex for catalytic functionality, but where said complex is only formed transiently during each catalytic cycle (for example, H. sapiens cytochrome P450 3A4 plus H. sapiens NADPH-cytochrome P450 reductase)

Included within the scope of the invention is the immobilisation of functional co-enzyme complexes (for example, NADPH-cytochrome P450 reductase/P450) in an array format. Thus an enzyme and its accessory protein may occupy the same location on an array.

In a second aspect, the invention provides a protein array comprising a surface having a plurality of spatially defined locations wherein at each location there are deposited at least two protein moieties characterised in that in that said protein moieties at each location act sequentially on a substrate of interest.

Each position in the pattern of an array of the second aspect can contain, for example, a sample of two or more protein types wherein said two or more proteins potentially act sequentially on a given small molecule but do not necessarily interact with each other (for example, H. sapiens cytochrome P450 3A4 plus H. sapiens glutathione S-transferase P1).

Also included in this aspect is a co-array of mixtures of phase 1 and phase 2 DMEs that mimic the in vivo situation more completely and enables the identity and relative proportions of the different metabolite products to be determined. This allows the full characterisation of the binding and metabolite profiles of a drug, particularly where the phase 1 DMEs catalyse the production of short lived electrophilic products which are then the substrates for the phase 2 DMEs. An example of a co-array format is a 96 or 384 well plate with a panel of P450s arrayed in columns and on the same plate a panel of drug conjugative enzymes arrayed in rows. In this way the pairings of the phase 1 and 2 relevant for metabolism of a particular drug can be rapidly determined. The co-arrays are typically in a form where the phase 1 DME is immobilised and the phase 2 DME is either immobilised or in solution phase; identification of metabolites is typically by LC-MS.

In an embodiment of the first and second aspects of the invention, at least one of protein moieties at each location on the protein array is capable of being membrane-associated or membrane-bound or has been modified to interact with a non-polar or amphipathic molecule.

For example, a hydrophobic peptide attached to the N- or C-terminus of a protein of interest and/or a native hydrophobic region, for example patch on the surface of the protein, is used to immobilise the proteins on the array surface through interaction with liposomes or microsomes encapsulated within a hydrogel matrix on the surface. Where a protein of interest is sufficiently lipophobic such that it cannot be prepared in a membrane-like preparation such as a detergent micelle, the enzyme can be modified to interact with the lipid or detergent molecules used to form the membrane-like preparation, for example by the addition of a hydrophobic tag or the insertion of a transmembrane domain from another protein (provided that these modifications do not alter the catalytic activity of the protein).

In another, preferred, embodiment the surface coating is a gel matrix, for example, a hydrogel polymer, such as agarose, polyurethane or polyacrylamide in which liposomes or microsomes are encapsulated such that each protein moiety interacts with said encapsulated liposome or microsome via a hydrophobic peptide positioned at the N- or C-terminus of each protein and/or a hydrophobic patch or region on, for example, the surface of each protein. The use of liposomes or microsomes on the array allows a transient interaction to take place or transient complex to be formed between the two or more proteins positioned at each location on the array during catalysis. This allows for the first time, arrays of co-operating proteins (for example P450 and NADPH-cytochrome P450 reductase) to be made.

In one embodiment of the first and second aspects of the invention the protein moieties on the array are derived from drug metabolising enzymes

However, the arrays of the first and second aspects of the invention are not limited to those carrying drug metabolising enzymes. The arrays of these aspects may comprise any proteins of interest which are capable of forming a transient complex or which act sequentially on a substrate of interest.

In a third aspect, the invention provides a protein array comprising a surface upon which are deposited at spatially defined locations at least two protein moieties characterised in that said protein moieties are derived from one or more DMEs. In an embodiment of this aspect, a DME may stand alone (without a partner in a complex) at each location of the array and be chemically activated, for example chemical activation of an immobilised P450 enzyme via the peroxide shunt pathway.

A protein array as defined herein is a spatially defined arrangement of protein moieties in a pattern on a surface. Preferably the protein moieties are attached to the surface either directly or indirectly. The attachment can be non-specific (for example, by physical absorption onto the surface or by formation of a non-specific covalent interaction). In a preferred embodiment the protein moieties are attached to the surface through a marker moiety or tag (for example, a hexa-hisitidine tag or a chemically attached molecule such as biotin) appended to each protein moiety. In one embodiment, the marker moiety or tag can be common to all protein moieties to be arrayed. In another preferred embodiment, the protein moieties can be incorporated into a vesicle or liposome which is immobilised in proximity to the surface for example by a gel matrix.

A surface as defined herein is a flat or contoured area that may or may not be coated/derivatised by chemical treatment. For example, the area can be a glass slide, one or more beads, for example a magnetic, derivatised and/or labelled bead as known in the art, a gold, silica or metal object, ceramic sol gels, polypropylene, polystyrene, gold or silica slides, polypropylene or polystyrene multi-well plates, or other porous surfaces such as nitrocellulose, PVDF, nylon or phosphocellulose membranes.

Where a bead is used, individual proteins, pairs of proteins or pools of variant proteins can be attached to an individual bead to provide the spatial definition or separation of the array. The beads can then be assayed separately, but in parallel, in a compartmentalised way, for example in the wells of a microtitre plate or in separate test tubes. These formats would be useful in, for example, “shotgun screening” to initially identify groups of proteins in which a protein of interest may exist; such groups are then separated and further investigated. This method is analogous to pooling methods known in the art of combinatorial chemistry.

Thus a protein array comprising a surface according to the invention can exist as a series of separate solid phase surfaces, such as beads carrying different proteins, the array being formed by the spatially defined pattern or arrangement of the separate surfaces in the experiment.

Preferably the surface has a surface coating which is capable of resisting non-specific protein absorption. The surface coating can be porous or non-porous in nature. In addition, in a preferred embodiment the surface coating provides a specific interaction with the marker moiety on each protein moiety either directly or indirectly (for example, through a protein or peptide or nucleic acid bound to the surface). Neutravidin-derivatised, dextran-hydrogel surfaces (XanTec, Muenster, Germany) can be used as the capture surface, although a variety of other surfaces can be used, as well as surfaces in microarray or microwell formats as known in the art.

In another embodiment, the individual members of the protein array each contain a peptide or polypeptide tag, for example a hexahistidine tag or a biotin carboxyl carrier protein derived tag, through which they can be immobilised, thereby minimising the risk of perturbing the function of the arrayed proteins through non-specific contact with the surface.

A protein moiety is a protein or a polypeptide and is typically encoded by a DNA sequence which is generally derived from a gene or a naturally occurring variant of the gene. The protein moiety can be derived from a recombinant or native source or it could be synthesised by ligation of a series of synthetic peptides which can contain non-natural amino acid residues and as such may not be directly encoded by a DNA sequence. The protein moiety can take the form of a protein directly encoded by a natural gene, or can comprise additional amino acids (not originally encoded by the DNA sequence from which it is derived) to facilitate attachment to the array or analysis in an assay.

Also included within the scope of the invention are arrays carrying protein moieties encoded by synthetic equivalents of a wild type gene (or a naturally occurring variant thereof) which encode the same amino acid sequence but which comprise one or more different codons to the wild type or mutant gene such that the synthetic DNA sequence would not map to the same chromosomal locus.

Whilst, for example, a set of DME proteins can be attached to the array via a binding protein or an antibody or a liposome or microsome which is capable of binding an invariant or common part of the individual proteins in the set, protein moieties according to the invention can also be proteins tagged (via the combination of the protein encoding DNA sequence with a tag encoding DNA sequence) at either the N- or C-terminus with a marker moiety to facilitate purification and/or attachment to the array.

In the third aspect of the invention, each position in the pattern of an array can contain, for example, either:
a sample of a single DME type (in the form of a monomer, dimer, trimer, tetramer or higher multimer) or
a sample of a single DME type bound to an interacting molecule (for example, nucleic acid molecule, antibody, other protein or small molecule. The interacting molecule may itself interact with further molecules. For example, one subunit of an heteromeric protein can be attached to the array and a second subunit or complex of subunits can be tethered to the array via interaction with the attached protein subunit. In turn the second subunit or complex of subunits can then interact with a further molecule, for example, a candidate drug or an antibody) or
a sample of a single DME type bound to a synthetic molecule (for example, peptide, chemical compound).

The proteins derived from the expression of more than one DNA sequence encoding a DME can be attached at a single position in an array for example, for the purposes of initial bulk screening of a sets of DMEs to determine those sets containing DMEs of interest.

In one embodiment of the invention a biotin tag attached to the DME protein is used to immobilise and purify the proteins on the array surface. However, the functionality of the array is independent of tag used. Alternative affinity tags to biotin tags (for example H is, FLAG, c-myc, VSV) can be used to enable purification and/or immobilisation of the cloned proteins. Also an expression host other than E. Coli can be used (for example, yeast, insect cells, mammalian cells) if required.

The present invention provides arrays carrying a collection of proteins which can represent all or a proportion of the drug metabolising enzymes of an organism. The individual proteins in said collection are purified in a folded conformation. In addition, the individual proteins are spatially separated and immobilised on a surface in an array format such that the folded state of the individual proteins is unlikely to be perturbed. Immobilisation of, for example, functional P450s in a spatially defined array enables multiplexed drug binding assays, enzymatic turn-over assays and cytotoxicity assays to be carried out, all in a miniaturised format, and offers a number of advantages over current state-of-the-art, solution phase methods.

By arraying out the DME proteins in a microtitre plate or on a microscope slide, many different proteins (hundreds or even thousands) can be assayed simultaneously using only small amounts of compound, thus enabling the simultaneous, quantitative functional analysis of large numbers of compounds against, for example, multiple cytochrome P450 proteins. In using an array format, all proteins are assayed together in the same experiment, thus reducing-sources of error due to differential handling of materials. Compared to individual solution phase assays, array-based assays are also very rapid to set up and perform. In addition, immobilisation of the proteins on a solid support facilitates binding assays which require unbound ligands to be washed away prior to measuring bound concentrations, a feature not available in solution based or single phase liquid assays. Immobilisation of the DMEs also means that a protein removal step is not required prior to high through-put mass spectrometric or HPLC analysis of the metabolites generated from turn-over of the ligands by the DMEs. Further, no clean-up step is required prior to cell based assays with the generated metabolites, thus enabling cytotoxicity assays to be performed on such metabolites, even where such metabolites are unstable and have a short half-life which effectively precludes their purification.

The array format allows the collection of drug metabolising enzymes to be interrogated with a range of functional assays in a highly parallel, quantitative manner to identify, for example, whether individual new chemical entities (NCEs) are inhibitors or substrates for any DME. Where an NCE is found to be a substrate for one or more DMEs, the array format also enables rapid, quantitative, and high throughput identification of the metabolites produced and also enables coupled cytotoxicity assays to be carried out with prior isolation or purification of said metabolites. The array format also allows the parallel quantitation of individual DME expression levels in, for example, drug-treated cells through competition assays; such assays involve an immobilised DME, the same DME in, for example, a tissue homogenate, and a labelled recognition agent, such as a fluorescently labelled antibody that is specific for the particular DME.

Preferably, the full complement, or a significant proportion of human DMEs are present on the arrays of the invention. Such an array can include (numbers in parenthesis currently described in the Swiss Prot database): all the human P450s (119), FMOs (5), UDP-glycosyltransferase (UGTs) (18), GSTs (20), sulfotransferases (SULTs) (6), N-acetyltransferases (NATs) (2), drug binding nuclear receptors (33) and drug transporter proteins (6). This protein list does not include those yet to be characterised from the human genome sequencing project, splice variants known to occur for the P450s that can switch substrate specificity or polymorphisms known to affect the function and substrate specificity of both the P450s and the phase 2 DMEs.

Usefully, DNA molecules encoding all known DMEs in one or more organisms are used to produce a set of protein moieties which are attached to the arrays of the invention. Optionally, the array can comprise a subset of DME proteins derived from a subset of DNA molecules.

The number of DME proteins attached to the arrays of the invention is determined by the number of DME coding sequences that are of sufficient experimental, commercial or clinical interest for one or more particular investigations. An array carrying a single DME would be of use to the investigator. However in practice and in order to take advantage of the suitability of such arrays for high throughput assays, it is envisaged that 1 to 10000, 1 to 1000, 1 to 500, 1 to 400, 1 to 300, 1 to 200, 1 to 100, 1 to 75, 1 to 50, 1 to 25, 1 to 10 or 1 to 5 DME encoding DNA molecules are represented by their encoded proteins on an array. Using current robotic spotting capabilities it is possible to increase spot density to include over 10,000 proteins per array. For example, an array comprising the H. sapiens cytochrome P450s CYP1A2, CYP2A6, CYP2B6, CYP2C8, CYP2C9*1, CYP2C9*2, CYP2C9*3, CYP2C19, CYP2D6, CYP2 μl, CYP3A4, and CYP3A5 would be useful in determining which, if any, of said P450s are responsible for metabolising a given small molecule. Alternatively, an array of the functional polymorphisms of H. sapiens cytochrome P450s CYP2C9, CYP2D6 and CYP3A4 would be useful in determining whether a given small molecule will be metabolised at different rates, or will give rise to different products, in the different ethnic groups likely to be sampled in a clinical trial.

The invention provides methods that by expression, purification and orientated immobilization, whilst retaining functionality, of the above proteins in array format enable multiplexed, high-throughput assays to establish the metabolite profile of, for example, a drug lead. Such assays include measurement of small molecule drug binding and the calculation of dissociation constants measured by radiometric, phosphor-imager, calorimetric, colorimetric, fluorescence (time resolved, polarization, resonance energy transfer), phosphorescence, surface plasmon resonance, chemiluminesence, light refraction or mass spectroscopic (MS) methods. Small molecule drug inhibition of enzymes (reversible or suicide) or enhancement of activity of enzymes can be detected by: the turn-over of fluorescent substrates, such as the conversion of dibenzyl fluorescein to fluorescein for P450 2C9 or benzyl resorufin to resorufin for P450 3A4; peroxide depletion assays when direct chemical activation of the P450s is used with the addition of cumene peroxide or hydrogen peroxide; measurement of formaldehyde generation using the Nash reagent during demethylation assays; thin layer or liquid chromatography (TLC or HPLC); and MS. Enzymatic drug turn-over and the production of metabolite products can be detected by peroxide depletion assays, thin layer and liquid chromatography, MS and nuclear magnetic resonance (NMR). Characterization of the possibly multiple metabolites produced during turn-over by the drug metabolizing enzymes can be made by MS (ES, FAB, MALDI), NMR, elemental analysis and absorbance spectra (infra-red, visible and ultra-violet). Comparisons can also be made with animal (for example, mouse and rat) DMEs, nuclear receptors and drug transport proteins regarding drug binding and turn-over to relate the in vitro studies with animal in vivo results.

The arrays of the present invention allow massively parallel analysis of DMEs, have a sensitivity of analysis at least comparable to existing methods and enable quantitative, comparative functional analysis of DMEs in a manner not previously possible.

The arrays are compatible with protein-protein, protein-nucleic acid, protein-ligand, or protein-small molecule interactions and post-translational modifications in situ i.e. on the array “on-chip”. Arrays according to the invention are spotting density independent. The array format used in the invention enables analysis to be carried out using small volumes of potentially expensive ligands or substrates. Information provided by parallel protein arrays according to the invention will be extremely valuable for drug discovery and pre-clinical analyses of candidate drugs.

In a fourth aspect, the invention provides a method of making a protein array comprising the steps of:

a) providing two or more drug metabolising enzymes of interest from either recombinant, native or synthetic sources;
b) depositing said proteins at spatially defined locations on a surface to give an array.

The method can be adapted to purify the DMEs on the array. Said drug metabolising enzymes are brought into contact with the array in admixture with other protein molecules and deposition on the array occurs with simultaneous purification of the protein moiety on the array via a tag incorporated in the protein moiety. This can be done by means of “surface capture” by which is meant the simultaneous purification and isolation of the protein moiety on the array via an incorporated tag.

In another embodiment the drug metabolising enzymes are deposited with other proteins from an expression host cell on a surface at spatially defined locations to give an array.

The DNA molecules which are expressed to produce the protein moieties of the array can be generated using techniques known in the art (for example see Current Protocols in Molecular Biology, Volume 1, Chapter 8, Edited by Ausubel et al.). It will be understood by those skilled in the art that the expression host need not be limited to E. coli—yeast, insect or mammalian cells can be used. Use of a eukaryotic host may be desirable where the protein under investigation is known to undergo post-translational modification such as glycosylation.

To make the array, clones can optionally be grown in microtiter plate format allowing parallel processing of samples in a format that is convenient for arraying onto slides or plate formats and which provides a high-throughput format. Protein expression is induced and clones are subsequently processed for arraying. This can involve purification of the proteins by affinity chromatography, or preparation of lysates ready for arraying onto a surface which is selective for the recombinant protein (‘surface capture’). Thus, the DNA molecules can be expressed as fusion proteins to give protein moieties tagged at either the N- or C-terminus with a marker moiety. As described herein, such tags can be used to purify or attach the proteins to the surface or the array. Optionally, the protein moieties are simultaneously purified from the expression host lysate and attached to the array by means of the marker moiety. The resulting array of proteins can then be used to assay the functions of all proteins in a parallel, and therefore high-throughput manner.

In a fifth aspect, the invention provides a method of making a protein array comprising the steps of:

a) providing proteins from either recombinant, native or synthetic sources incorporated in purified or partially purified membrane or membrane-like preparations (for example, a microsomal preparation or a liposome formed with a detergent)
b) arraying said proteins by encapsulation of said membrane or membrane-like preparations into a gel matrix (for example, agarose, polyurethane, or polyacrylamide) which is deposited on the surface.

In order for the proteins of this aspect of the invention to be incorporated in purified or partially purified membrane or membrane-like preparations, it is necessary that they are either capable in their native state of being membrane-associated or membrane bound proteins or have been modified to interact with a non-polar molecule such as a membrane lipid or an amphipathic molecule such as a detergent. Such modification may be carried out by methods known in the art, for example, by the addition of a hydrophobic tag to the protein (for example, altering the coding sequence for the protein to incorporate a tag comprising a string of hydrophobic amino acids, for example at the N or C terminus of the protein).

In a sixth aspect, the invention provides a method of making an array of drug metabolising enzymes comprising the steps of:

a) providing drug metabolising enzymes from either recombinant, native or synthetic sources in the form of purified or partially purified-membrane or membrane-like-preparations (for example, a microsome or a lipsome)
b) arraying said drug metabolising enzymes either by deposition of said membrane or membrane-like preparations onto a suitable surface capable of capturing the membranes (for example, γ-aminopropyl silane) or by encapsulation of said membrane or membrane-like preparations into a gel matrix (for example, agarose, polyurethane, or polyacrylamide) which is deposited on the surface.

In the fifth and sixth aspects one or more of said membrane or membrane-like preparations contains two or more different proteins which are capable of forming a complex with each other, for example where said complex is transiently formed, or contains two or more different proteins which act sequentially on a substrate of interest.

In a seventh aspect, the invention provides a method of simultaneously determining the relative properties of members of a set of DME protein moieties, comprising the steps of: bringing an array as herein described said array into contact with one or more test substances, and observing the interaction of said test substances with the set members on the array.

In one embodiment, the invention provides a method of screening a set of DME protein moieties for compounds (for example, a small organic molecule) which enhance, restore or disrupt function of a protein, which can reveal compounds with therapeutic advantages or disadvantages.

In other embodiments the test substance can be:
a protein for determining relative protein-protein interactions within a set of protein moieties derived from related DNA molecules
a nucleic acid molecule for determining relative protein-DNA or protein-RNA interactions
a ligand for determining relative protein-ligand interactions

Results obtained from the interrogation of arrays of the invention can be quantitative (for example, measuring binding or catalytic constants K_{D} & K_{M}), semi-quantitative (for example, normalising amount bound against protein quantity) or qualitative (for example, functional vs. non-functional). By quantifying the signals for replicate arrays where the ligand is added at several (for example, two or more) concentrations, both the binding affinities and the active concentrations of protein in the spot can be determined. This allows comparison of DMEs with each other. This level of information has not been obtained previously from arrays. Exactly the same methodology can be used to measure binding of drugs to arrayed proteins.

For example, quantitative results, K_{D} and Bₘₐₓ, which describe the affinity of the interaction between ligand and protein and the number of binding sites for that ligand respectively, can be derived from protein array data. Briefly, either quantified or relative amounts of ligand bound to each individual protein spot can be measured at different concentrations of ligand in the assay solution. Assuming a linear relationship between the amount of protein and bound ligand, the (relative) amount of ligand bound to each spot over a range of ligand concentrations used in the assay can be fitted to equation 1, rearrangements or derivations.

Bound ligand=Bₘₐₓ/((K_{D}[L]+1) (Equation 1)
[L]=concentration of ligand used in the assay

In an eighth aspect, the invention provides a method of expressing and purifying DME enzymes, comprising the steps of:

a) expressing a DMB of interest in a host cell (for example E. coli, such as XL-10 gold);
b) subjecting said host cell to conditions suitable to lyse the cell (for example, after pelleting the cell culture, by conventional treatment with lysis buffer, MgCl₂ and DNaseI);
c) obtaining a membrane associated cell fraction from the lysed cell (for example by centrifugation at around 4000 rpm to form a pellet);
d) solubilising said membrane associated cell fraction by the addition of a detergent (for example, a nonionic detergent, such as 0.3% (v/v) Igepal CA-630 in a suitable buffer);
e) after an incubation period sufficient to solubilise the DME protein contained in said membrane associated cell fraction, performing a further centrifugation step (for example, at around 10,000 g) to produce a supernatant containing said DME protein;
f) subjecting said supernatant to chromatography to purify said DME protein (for example where the DME protein has been modified to incorporate a hexahistidine tag by use of a metal affinity chromatography matrix such as Talon resin (Clontech) and/or a Ni-NTA agarose matrix (Qiagen).

The method of this aspect uses detergents to solubilise DME proteins of interest and, as a result, does not require an ultra-centrifugation step. All previously reported P450 purification approaches have required an ultracentrifugation step which means that it is difficult to perform P450 purifications in a multiplexed manner. Thus this method is particularly applicable to the production of proteins for protein arrays according to the invention. An embodiment of this method is described in Example 4 herein.

Arrays according to the invention can be used in various types of analysis. Several non-exhaustive and non-limiting illustrations of such use now follow:

A first use of the arrays as described herein is in providing a high throughput, quantitative tool for the early evaluation of whether ‘hit series’ or ‘lead series’ compounds or drug candidates are substrates or inhibitors of phase 1 DMEs. For example, the collection of compounds which are identified from an initial high throughput screen against a single protein target can be evaluated for their ability to act as substrates or inhibitors against an array of H. sapiens cytochrome P450s including CYP1A2, CYP2A6, CYP2B6, CYP2C8, CYP2C9*1, CYP2C9*2, CYP2C9*3, CYP2C19, CYP2D6, CYP2E1, CYP3A4, and CYP3A5. This is possible since the array-based assays require only small amounts of each compound, in an unlabelled form, and are therefore compatible with the scale of compound synthesis usually available even in primary, unscreened compound libraries. The array-based assays can be in a number of different formats, including competitive binding assays with a known, radiolabelled inhibitor (for example, ³H-ketoconazole for CYP3A4), kinetic analysis of the effect on turnover rate for known, fluorescent substrates (for example, dibenzyl fluorescein for CYP2C9 or CYP3A4), and direct high throughput analysis of product formation by LC-MS methods. The data generated through use of such an array will be useful in, amongst others, predicting potential drug-drug interactions, and in lead selection/optimisation.

A second use of arrays of DMEs described herein is in providing a high throughput, quantitative tool to examine gender differences in drug metabolism. It has been shown that male and female rats express P450 isoforms differently, due to different profiles of hormone secretion (Shapiro et al., 1995). For example, it was found that women metabolise the corticosteroid methyl-prednisolone more quickly than men and that women were more sensitive to the steroids effects as measured by serum cortisol concentrations and lymphocyte count (Lew et al., 1993). However for prednisolone (where a methyl group is removed) no marked difference in the metabolism rate between men and women was observed (Magee et al., 2001) indicating that one could perform structure activity studies (SAR) to abolish gender differences of drugs. It is likely that gender differences will be required to be examined in the future for drug development and regulatory authority approval (www.fda.gov/womens/executive.html). An application of the technology described here is to develop male and female DME protein arrays since it is known that women and men can express a different panel of P450s or many of the same ones at different levels. Alternatively a single array can highlight the potential for gender differences in drug metabolism.

A third use of arrays of DMEs according to the invention is in providing a high throughput, quantitative tool to examine ethnicity-related differences in drug metabolism and toxicity, making possible tailored drug treatment for various ethnic groups. For example it is known that there are large differences in the frequency of occurrence of various alleles in P450s 2C9, 2D6 and 3A4 between different ethnic groups (see Tables 1, 2 and 3). These alleles have the potential to affect enzyme kinetics, substrate specificity, regio-selectivity and, where multiple products are produced, product profiles. Arrays of proteins described in this disclosure allow a more detailed examination of these differences for a particular drug and will be useful in predicting potential problems and also in effectively planning the population used for clinical trials.

TABLE 1
P450 2D6 Allele Frequency

| | | | Allele | Ethnic | Study | |
|---|---|---|---|---|---|---|
| P450 | Allele | Mutation | Frequency | Group | Group | Reference |
| 2D6 | *1 | W.T. | 26.9% | Chinese | 113 | (1) |
| | | | 36.4% | German | 589 | (2) |
| | | | 36% | Caucasian | 195 | (3) |
| | | | 33% | European | 1344 | (4) |
| 2D6 | *2 | R296C; | 13.4% | Chinese | 113 | (1) |
| | | S486T | 32.4% | German | 589 | (2) |
| | | | 29% | Caucasian | 195 | (3) |
| | | | 27.1% | European | 1344 | (4) |
| 2D6 | *3 | Frameshift | 2% | German | 589 | (2) |
| | | | 1% | Caucasian | 195 | (3) |
| | | | 1.9% | European | 1344 | (4) |
| 2D6 | *4 | Splicing | 20.7% | German | 589 | (2) |
| | | defect | 20% | Caucasian | 195 | (3) |
| | | | 16.6% | European | 1344 | (4) |
| | | | 1.2% | Ethiopian | 115 | (5) |
| 2D6 | *5 | Deletion | 4% | Caucasian | 195 | (3) |
| | | | 6.9% | European | 1344 | (4) |
| 2D6 | *6 | Splicing | 0.93% | German | 589 | (2) |
| | | defect | 1.3% | Caucasian | 195 | (3) |
| 2D6 | *7 | H324P | 0.08% | German | 589 | (2) |
| | | | 0.3% | Caucasian | 195 | (3) |
| | | | 0.1% | European | 1344 | (4) |
| 2D6 | *9 | K281del | 2% | Caucasian | 195 | (3) |
| | | | 2.7% | European | 1344 | (4) |
| 2D6 | *10 | P34S; | 50.7% | Chinese | 113 | (1) |
| | | S486T | 1.53% | German | 589 | (2) |
| | | | 2% | Caucasian | 195 | (3) |
| | | | 1.5% | European | 1344 | (4) |
| | | | 8.6% | Ethiopian | 115 | (5) |
| 2D6 | *12 | G42R; | 0% | German | 589 | (2) |
| | | R296C; | 0.1% | European | 1344 | (4) |
| | | S486T | | | | |
| 2D6 | *14 | P34S; | 0.1% | European | 1344 | (4) |
| | | G169R; | | | | |
| | | R296C; | | | | |
| | | S486T | | | | |
| 2D6 | *17 | T107I; | 0% | Caucasian | 195 | (3) |
| | | R296C; | 0.1% | European | 1344 | (4) |
| | | S486T | 9% | Ethiopian | 115 | (5) |
| | | | 34% | African | 388 | (6) |

All other P450 allelic variants occur at a frequency of 0.1% or less (4).

TABLE 2
P450 2C9 Allele Frequency

| | | Muta- | Allele | Ethnic | Study | |
|---|---|---|---|---|---|---|
| P450 | Allele | tion | Frequency | Group | Group | Reference |
| 2C9 | *1 | W.T. | 62% | Caucasian | 52 | (7) |
| 2C9 | *2 | R144C | 17% | Caucasian | 52 | (7) |
| 2C9 | *3 | I359L | 19% | Caucasian | 52 | (7) |
| 2C9 | *4 | I359T | x% | Japanese | x | (8) |
| 2C9 | *5 | D360E | 0% | Caucasians | 140 | (9) |
| | | | 3% | African- | 120 | (9) |
| | | | | Americans | | |
| 2C9 | *7 | Y358C | x% | | x | Swiss Prot |

TABLE 3
P450 3A4 Allele Frequency

| | | | Allele | Ethnic | Study | |
|---|---|---|---|---|---|---|
| P450 | Allele | Mutation | Frequency | Group | Group | Reference |
| 3A4 | *1 | W.T. | >80% | | X | |
| 3A4 | *2 | S222P | 2.7% | Caucasian | X | (10) |
| | | | 0% | African | x | (10) |
| | | | 0% | Chinese | x | (10) |
| 3A4 | *3 | M445T | 1% | Chinese | X | (10) |
| | | | 0.47% | European | 213 | (11) |
| | | | 4% | Caucasian | 72 | (12) |
| 3A4 | *4 | I118V | 2.9% | Chinese | 102 | (13) |
| 3A4 | *5 | P218R | 2% | Chinese | 102 | (13) |
| 3A4 | *7 | G56D | 1.4% | European | 213 | (11) |
| 3A4 | *8 | R130Q | 0.33% | European | 213 | (11) |
| 3A4 | *9 | V170I | 0.24% | European | 213 | (11) |
| 3A4 | *10 | D174H | 0.24% | European | 213 | (11) |
| 3A4 | *11 | T363M | 0.34% | European | 213 | (11) |
| 3A4 | *12 | L373F | 0.34% | European | 213 | (11) |
| 3A4 | *13 | P416L | 0.34% | European | 213 | (11) |
| 3A4 | *15 | R162Q | 4% | African | 72 | (12) |
| 3A4 | *17 | F189S | 2% | Caucasian | 72 | (12) |
| 3A4 | *18 | L293P | 2% | Asian | 72 | (12) |
| 3A4 | *19 | P467S | 2% | Asian | 72 | (12) |

A fourth use of the arrays of the invention is in providing a high throughput, quantitative tool to examine differences in drug metabolism between two or more mammalian species, for example, rodents (for example, rats) and humans. Currently all pre-clinical, whole organism toxicology and metabolism studies are carried out on rats. However, whilst there is typically strong overall sequence homology between the rat and human isoforms of any given DMF, there may be subtle functional differences between the isoforms which could affect the distribution or identity of specific metabolites produced as a result of turn-over by the rat or human DMEs. An array containing both human and rat isoforms of phase I DMs (for example, H. sapiens CYP2C9, CYP2D6, CYP3A4 and Rattus norvegicus CYP2C9, CYP2D6, CYP3A4) thus provides a high thoughput, quantitative screening tool to identify any species-related differences in drug metabolism and will therefore enable useful additional data to be obtained on drug metabolism and toxicity in advance of clinical trials involving humans.

A fifth use of the arrays of the invention is in providing a high throughput, quantitative tool to examine the possible cytotoxicity of drug metabolites, including those that are short-lived. Thus an array of phase 1 DMEs can be overlaid with cells that act as reporters in a cytotoxicity assay such that any metabolites produced by the phase 1 DMEs can be assayed for cytotoxic effects in situ, i.e. without isolation or purification of the metabolite itself.

A sixth use of the arrays of the invention is in providing a high throughput tool to define and quantitate metabolism pathways for small molecules. Thus an array comprising a matrix of phase 1 and phase 2 DMEs (for example, P450 CYP2C9, CYP2D6 and CYP3A4, each co-arrayed with a glutathione S-transferase, a glucuronyl transferase and a sulphotransferase) can be used to evaluate which combinations of P450 and drug conjugating enzyme are responsible for metabolism of a particular drug and also which combinations might give rise to toxic metabolites. For example, the primary metabolite of the pain-killer paracetamol is detoxified by glutathione S-transferase, whereas the primary metabolite of the drug tamoxifen is detoxified by glucuronidation but is converted to a toxic adduct by sulphate transfer.

A seventh use of the arrays of the invention is in ‘hit series’ evaluation and lead optimisation when the DMEs are drug targets in their own right For example, oltipraz (Sofowora et al., 2001) is a currently undergoing clinical evaluation as a cancer chemopreventative agent and is a P450 1 A2 inhibitor. Thus, an array of DMEs provides a high thoughput method to screen compounds (hit series, lead series and drug candidates) for selectivity in their ability to bind and inhibit individual DMEs.

An eighth use of the arrays as described herein is in providing a high throughput, quantitative tool for the evaluation of drug-induction of P450 expression level. This is often difficult to carry out accurately and yet drug-induction of P450 expression is responsible for many adverse drug-drug interactions and the ability to quantitate this effect simply and rapidly would be very useful. Thus an array of immobilised P450s CYP1A2, CYP2C9, CYP2D6 and CYP3A4 can be used in a competitive binding assays to assess the relative expression levels of the equivalent P450s in healthy and drug-treated cells. Here, the assays involve use of, for example, a dye-labelled antibody which can bind to either immobilised P450 or to P450 in a crude tissue homogenate; the amount of antibody bound to the immobilised P450 and thus be used to quantitate the expression levels of the P450 in the healthy and drug treated tissue homogenates.

A ninth use of the arrays as described herein is in providing a high throughput, quantitative tool to analyse the effects of mutation on the activity of a given DME. For example, cytochrome CYP2C9 could be mutated using directed evolution approaches and an array of the resultant DME mutant collection could be screened for either increased catalytic efficiency or changes in substrate specificity. This will be of use to the chemical industry to develop more efficient or novel chemical synthesis routes. The advantage of this approach compared to phage or cell (Joo et al., 1999) selection, is that diversity would not be lost during the selection and amplification process. This is similar to the concepts behind affinity and selectivity maturation of antibodies using antibody arrays (de Wildt et al., 2000).

Preferred features of each aspect of the invention are as defined for each other aspect, mutatis mutandis.

Further features and details of the invention will be apparent from the following description of DME protein moiety arrays, methods of constructing such protein arrays, and their use in accordance with the invention which is given by way of example and with reference to the accompanying drawings, and which are not intended to limit the scope of the invention in any way.

### EXAMPLES

### Example 1

### Cloning of Wild Type H. sapiens Cytochrome P450 Enzymes CYP2C9, CYP2D6 and CYP3A4

The human cytochrome p450s have a conserved region at the N-terminus, this includes a hydrophobic region which facilitates lipid association, an acidic or ‘stop transfer’ region, which stops the protein being fed further into the membrane, and a partially conserved proline repeat. Three versions of the p450s were produced with deletions up to these domains, the N-terminal deletions are shown below.

| Construct | Version | N-terminal Deletion |
|---|---|---|
| T009-C2 3A4 | Proline | -34 AA |
| T009-C1 3A4 | Stop Transfer | -25 AA |
| T009-C3 3A4 | Hydrophobic peptide | -13 AA |
| T015-C2 2C9 | Proline | -28 AA |
| T015-C1 2C9 | Stop Transfer | -20 AA |
| T015-C3 2C9 | Hydrophobic peptide | -0 AA |
| T017-C1 2D6 | Proline | -29 AA |
| T017-C2 2D6 | Stop Transfer | -18 AA |
| T017-C3 2D6 | Hydrophobic peptide | -0 AA |

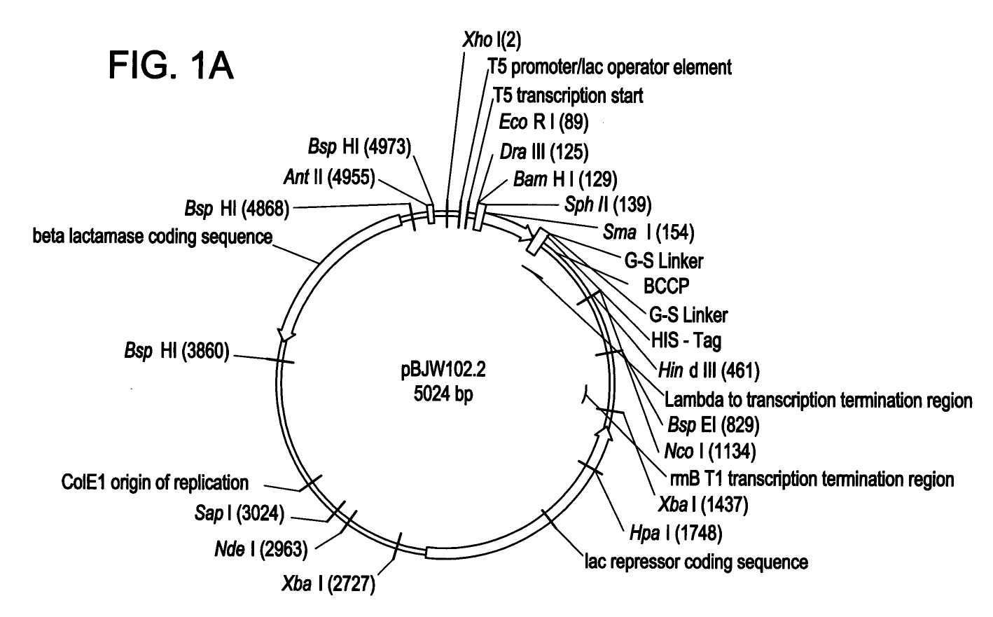

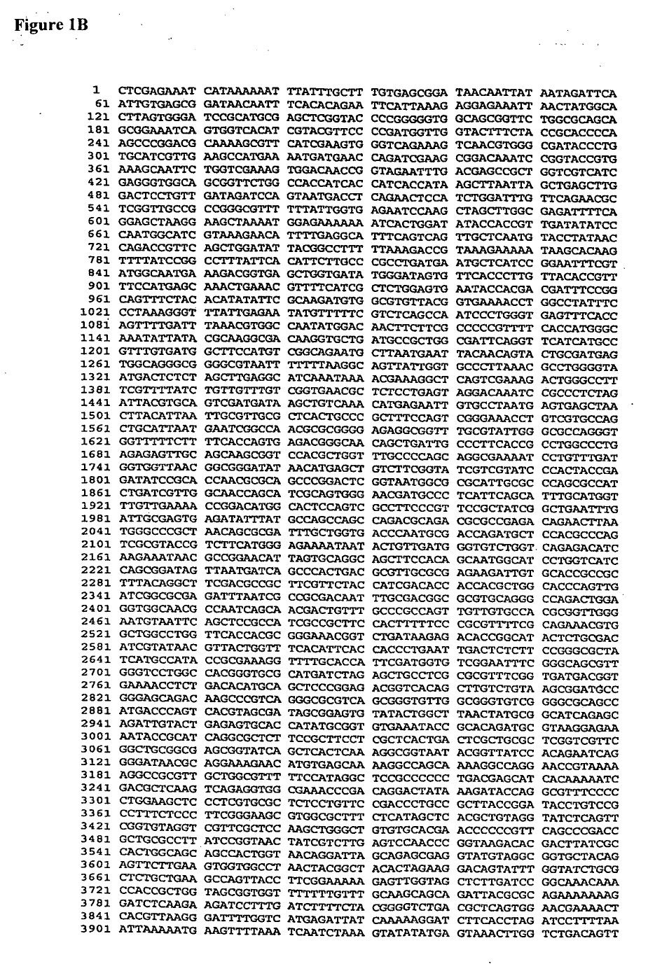

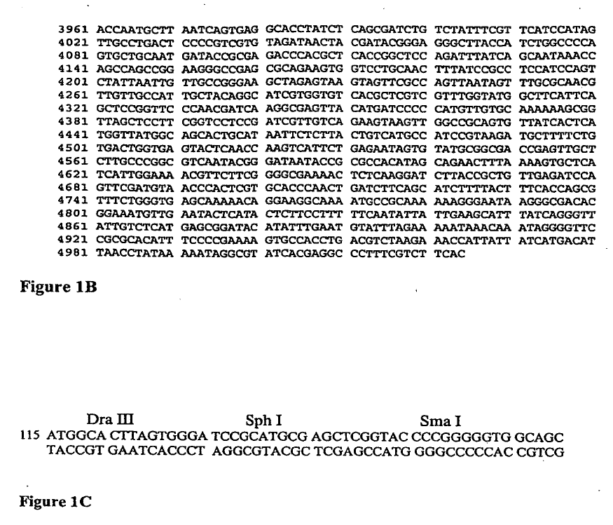

The human CYP2D6 was amplified by PCR from a pool of brain, heart and liver cDNA libraries (Clontech) using specific forward and reverse primers (T017F and T017R). The PCR products were cloned into the pMD004 expression vector, in frame with the N-terminal His-BCCP tag and using the Not1 restriction site present in the reverse primer. To convert the CYP2D6 for expression in the C-terminal tag vector pBJW102.2 (FIGS. 1A&B), primers were used which incorporated an Sfi1 cloning site at the 5′ end and removed the stop codon at the 3′ to allow in frame fusion with the C-terminal tag. The primers T017CR together with either T017CF1, T017CF2, or T017CF3 allowed the deletion of 29, 18 and 0 amino acids from the N-terminus of CYP2D6 respectively.

Primer sequences are as follows:

| | |
|---|---|
| T017F: | 5′-GCTGCACGCTACCCACCAGGCCCCCTG-3′ |
| T017R: | 5′-TTGCGGCCGCTCTTCTACTAGCGGGGCACAGCACAAAGC |
| | TCATAG-3′ |
| T017CF1: | 5′-TATTCTCACTGGCCATTACGGCCGCTGCACGCTACCCAC |
| | CAGGCCCCTG-3′ |
| T017CF2: | 5′-TATTCTCACTGGCCATTACGGCCGTGGACCTGATGCACC |
| | GGCGCCAACGCTGGGCTGCACGCTACCCACCAGGCCCCCTG- |
| | 3′ |
| T017CF3: | 5′-TATTCTCACTGGCCATTACGGCCATGGCTCTAGAAGCAC |
| | TGGTGCCCCTGGCCGTGATAGTGGCCATCTTCCTGCTCCTGG |
| | TGGACCTGATGCACCGGCGCCAACGC-3′ |
| T017CR: | 5′-GCGGGGCACAGCACAAAGCTCATAGGG-3′ |

PCR was performed in a 50 μl volume containing 0.5 μM of each primer, 125-250 μM dNTPs, 5 ng of template DNA, 1× reaction buffer, 1-5 units of polymerase (Pfu, Pwo, or ‘Expand long template’ polymerase mix), PCR cycle=95° C. 5 minutes, 95° C. 30 seconds, 50-70° C. 30 seconds, 72° C. 4 minutes×35 cycles, 72° C. 10 minutes, or in the case of Expand 68° C. was used for the extension step. PCR products were resolved by agarose gel electrophoresis, those products of the correct size were excised from the gel and subsequently purified using a gel extraction kit. Purified PCR products were then digested with either Sfi1 or Not1 and ligated into the prepared vector backbone (FIG. 1C). Correct recombinant clones were determined by PCR screening of bacterial cultures. Western blotting and by DNA sequence analysis.

CYP3A4 and CYP2C9 were cloned from cDNA libraries by a methodology similar to that of CYP2D6. Primer sequences to amplify CYP3A4 and CYP2C9 for cloning into the N-terminal vectors are as follows;

Primer wequences to amplify CYP3A4 and CYP2C9 for
cloning into the N-terminal vectores are as
follows;

| | |
|---|---|
| 2C9 | |
| T015F: | 5′-CTCCCTCCTGGCCCCACTCCTCTCCCAA-3′ |
| T015R: | 5′-TTTGCGGCCGCTCTTCTATCAGACAGGAATGAAGCACAG |
| | CCTGGTA-3′ |
| 3A4 | |
| T009F: | 5′-CTTGGAATTCCAGGGCCCACACCTCTG-3′ |
| T009R: | 5′-TTTGCGGCCGCTCTTCTATCAGGCTCCACTTACGGTGCC |
| | ATCCCTTGA-3′ |

Primers to convert the N-terminal clones for
expression in the C-terminal tagging vector
are as follows:

| | |
|---|---|
| 3A4 | |
| T009CF1: | 5′-TATTCTCACTGGCCATTACGGCCTATGGAACCCATTCAC |
| | ATGGACTTTTTAAGAAGCTTGGAATTCCAGGGCCCACACCTC |
| | TG-3′ |
| T009CF2: | 5′-TATTCTCACTGGCCATTACGGCCCTTGGAATTCCAGGGC |
| | CCACACCTCTG-3′ |
| T009CF3: | 5′-TATTCTCACTGGCCATTACGGCCCCTCCTGGCTGTCAGC |
| | CTGGTGCTCCTCTATCTATATGGAACCCATTCACATGGACTT |
| | TTTAGG-3′ |
| T009CR: | 5′-GGCTCCACTTACGGTGCCATCCCTTGAC-3′ |
| 2C9 | |
| T015CF1: | 5′-TATTCTCACTGGCCATTACGGCCAGACAGAGCTCTGGGA |
| | GAGGAAAACTCCCTCCTGGCCCCACTCCTCTCCCAG-3′ |
| T015CF2: | 5′-TATTCTCACTGGCCATTACGGCCCTCCCTCCTGGCCCCA |
| | CTCCTCTCCCAG-3′ |
| T015CR: | 5′-GACAGGAATGAAGCACAGCTGGTAGAAGG-3′ |

Primers to convert the N-terminal clones for expression in the C-terminal tagging vector are as follows:

| Primers to convert the N-terminal clones for expression in the | |
|---|---|
| C-terminal tagging vector are as folows: | |
| 3A4 | |
| T009CF1: | 5′-TATTCTCACTGGCCATTACGGCCTATGGAACCCATTCACATGGACTTTTTA |
| | AGAAGCTTGGAATTCCAGGGCCCACACCTCTG-3′ |
| T009CF2: | 5′-TATTCTCACTGGCCATTACGGCCCTTGGAATTCCAGGGCCCACACCTCTG-3′ |
| T009CF3: | 5′-TATTCTCACTGGCCATTACGGCCCCTCCTGGCTGTCAGCCTGGTGCTCCTCTATCTAT |
| | ATGGAACCCATTCACATGGACTTTTTAGG-3′ |
| T009CR: | 5′-GGCTCCACTTACGGTGCCATCCCTTGAC-3′ |
| 2C9 | |
| T015CF1: | 5′-TATTCTCACTGGCCATTACGGCCAGACAGAGCTCTGGGAGAGGAAAACTCCCTC |
| | CTGGCCCCACTCCTCTCCCAG-3′ |
| T015CF2: | 5′-TATTCTCACTGGCCATTACGGCCCTCCCTCCTGGCCCCACTCCTCTCCCAG-3′ |
| T015CR: | 5′-GACAGGAATGAAGCACAGCTGGTAGAAGG-3′ |

The full length or Hydrophobic peptide (C3) version of 2C9 was produced by inverse PCR using the 2C9-stop transfer clone (C1) as the template and the following primers:

2C9-hydrophobic-peptide-F:
5′-CTCTCATGTTTGCTTCTCCTTTCACTCTGGAGACAGCGCTCTGGGAG
AGGAAAACTC-3′
2C9-hydrophobic-peptide-R:
5′-ACAGAGCACAAGGACCACAAGAGAATCGGCCGTAAGTGCCATAGTTA
ATTTCTC-3′

### Example 2

### Cloning of NADPH-Cytochrome P450 Reductase

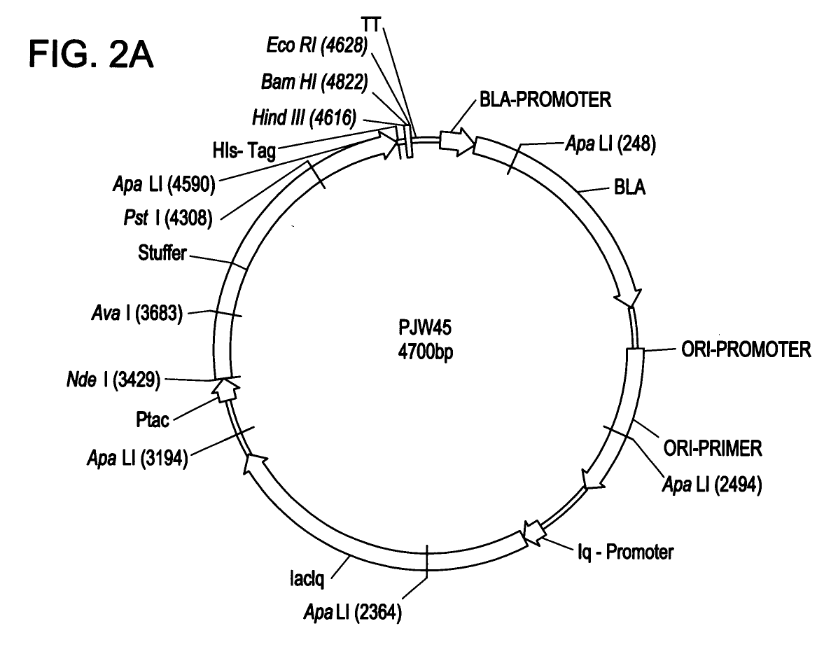

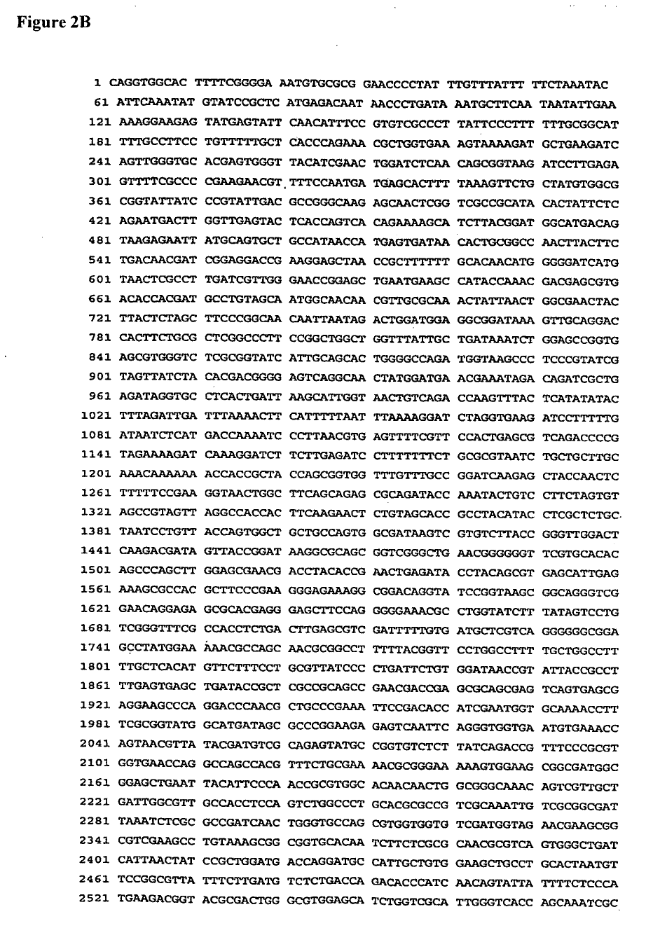

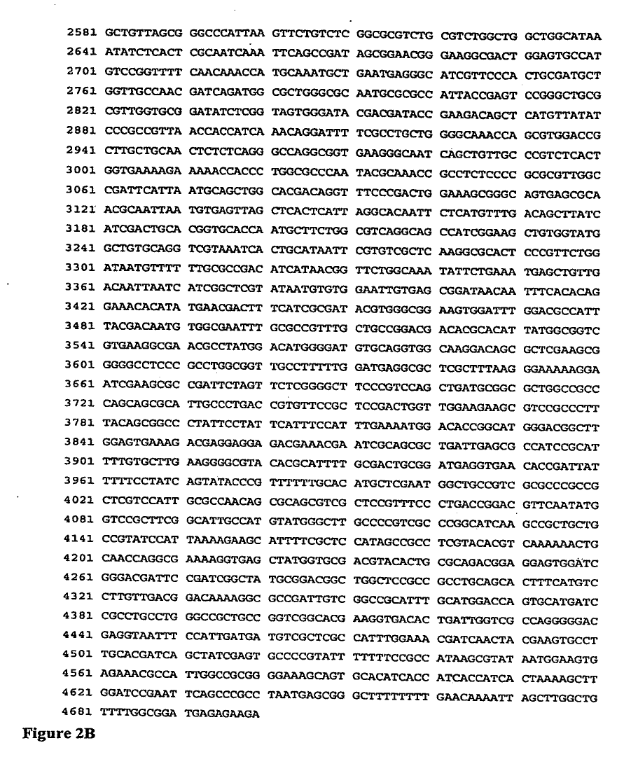

NADPH-cytochrome P450 reductase was amplified from fetal liver cDNA (Clontech), the PCR primers (NADPH reductase F1 5′-GGATCGACATATGGGAGACTCCCACGTGGACAC-3′; NADPH reductase R1 5′-CCGATAAGCTTATCAGCTCCACACGTCCAGGGAG-3′] incorporated a Nde I site at 5′ and a Hind III site at the 3′ of the gene to allow cloning. The PCR product was cloned into the pJW45 expression vector (FIGS. 2A&B)), two stop codons were included on the reverse primer to ensure that the His-tag was not translated. Correct recombinant clones were determined by PCR screening of bacterial cultures, and by sequencing.

### Example 3

### Cloning of Polymorphic Variants of H. sapiens Cytochrome P450s CYP2C9, CYP2D6 and CYP3A4

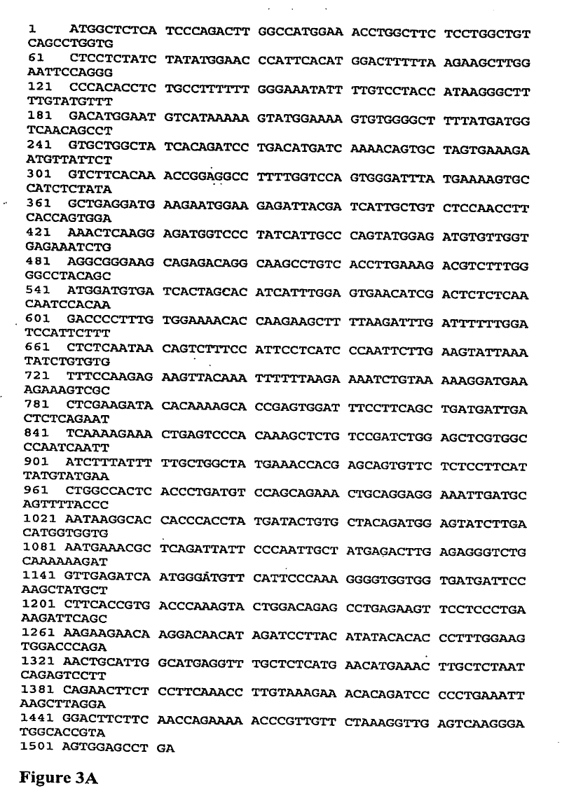

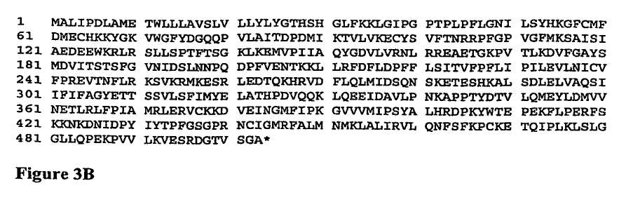

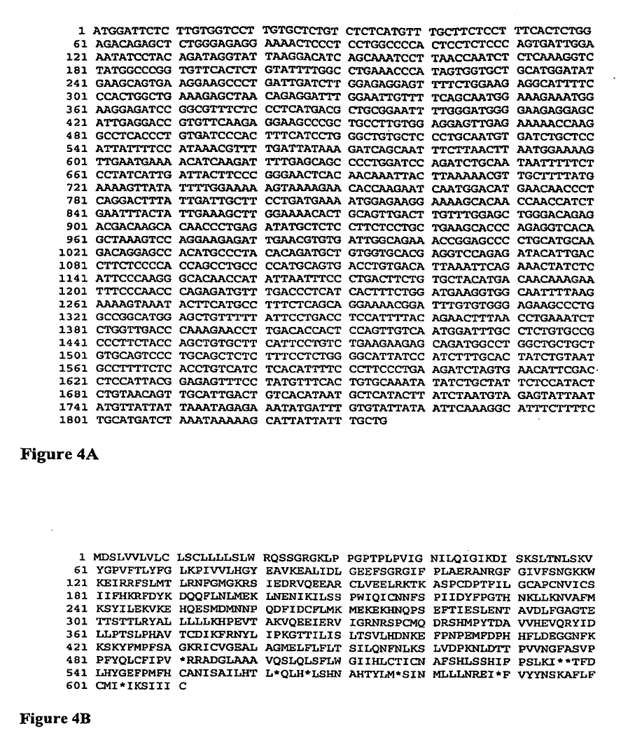

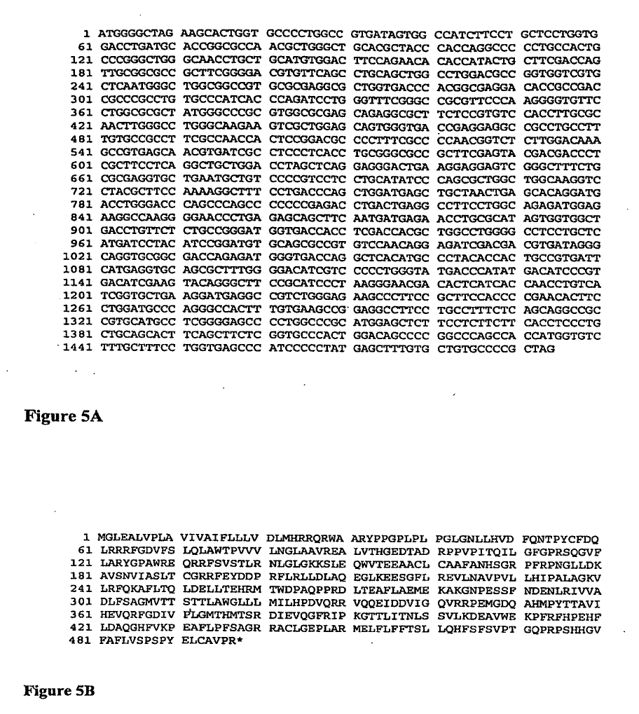

Once the correct wild-type CYP450s FIGS. 3, 4, & 5) were cloned and verified by sequence analysis the naturally occurring polymorphisms of 2C9, 2D6 and 3A4 shown in Table 4 were created by an inverse PCR approach (except for CYP2D6*10 which was amplified and cloned as a linear PCR product in the same way as the initial cloning of CYP2D6 described in Example 1). In each case, the forward inverse PCR primer contained a 1 bp mismatch at the 5′ position to substitute the wild type nucleotide for the polymorphic nucleotide as observed in the different ethnic populations.

TABLE 4
Polymorphic forms of P450 2C9, 2D6 and 3A4 cloned

| Cytochrome | |
|---|---|
| P450 polymorphism | Encoded amino acid subsitutions |
| CYP2C9*1 | wild-type |
| CYP2C9*2 | R144C |
| CYP2C9*3 | I359L |
| CYP2C9*4 | I359T |
| CYP2C9*5 | D360E |
| CYP2C9*7 | Y358C |
| CYP2D6*1 | wild-type |
| CYP2D6*2 | R296C, S486T |
| CYP2D6*9 | K281del |
| CYP2D6*10 | P34S, S486T |
| CYP2D6*17 | T107I, R296C, S486T |
| CYP3A4*1 | wild-type |
| CYP3A4*2 | S222P |
| CYP3A4*3 | M445T |
| CYP3A4*4 | I118V |
| CYP3A4*5 | P218R |
| CYP3A4*15 | R162Q |

The following PCR primers were used.

| | |
|---|---|
| CYP2C9*2F: | 5′-TGTGTTCAAGAGGAAGCCCGCTG-3′ |
| CYP2C9*2R: | 5′-GTCCTCAATGCTGCTCTTCCCCATC-3′ |
| CYP2C9*3F: | 5′-CTTGACCTTCTCCCCACCAGCCTG-3′ |
| CYP2C9*3R: | 5′-GTATCTCTGGACCTCGTGCACCAC-3′ |
| CYP2C9*4F: | 5′-CTGACCTTCTCCCCACCAGCCTG-3′ |
| CYP2C9*4R: | 5′-TGTATCTCTGGACCTCGTGCAC-3′ |
| CYP2C9*5F: | 5′-GCTTCTCCCCACCAGCCTGC-3′ |
| CYP2C9*5R: | 5′-TCAATGTATCTCTGGACCTCGTGC-3′ |
| CYP2C9*7F | 5′-GCATTGACCTTCTCCCCACCAGC-3′ |
| CYP2C9*7R: | 5′-CACCACGTGCTCCAGGTCTCTA-3′ |
| CYP2D6*10AF1: | 5′-TATTCTCACTGGCCATTACGGCCGTGGACCTGAT |
| | GCACCGGCGCCAACGCTGGGCTGCACGCTACTCACCA |
| | GGCCCCCTGC-3′ |
| CYP2D6*10AR1: | 5′-GCGGGGCACAGCACAAAGCTCATAGGGGGATGGG |
| | CTCACCAGGAAAGCAAAG-3′ |
| CYP2D6*17F: | 5′-TCCAGATCCTGGGTTTCGGGC-3′ |
| CYP2D6*17R: | 5′-TGATGGGCACAGGCGGGCGGTC-3′ |
| CYP2D6*9F: | 5′-GCCAAGGGGAACCCTGAGAGC-3′ |
| CYP2D6*9R: | 5′-CTCCATCTCTGCCAGGAAGGC-3′ |
| Cyp3A4*2F: | 5′-CCAATAACAGTCTTTCCATTCCTC-3′ |
| CYP3A4*2R: | 5′-GAGAAAGAATGGATCCAAAAAATC-3′ |
| CYP3A4*3F: | 5′-CGAGGTTTGCTCTCATGACCATG-3′ |
| CYP3A4*3R: | 5′-TGCCAATGCAGTTTCTGGGTCCAC-3′ |
| CYP3A4*4F: | 5′-GTCTCTATAGCTGAGGATGAAG-3′ |
| Cyp3A4*4R: | 5′-GGCACTTTTCATAAATCCCACTG-3′ |
| CYP3A4*5F: | 5′-GATTCTTTCTCTCAATAACAGTC-3′ |
| CYP3A4*5R: | 5′-GATCCAAAAAATCAAATCTTAAA-3′ |
| CYP3A4*15F: | 5′-AGGAAGCAGAGACAGGCAAGC-3′ |
| CYP3A4*15R: | 5′-GCCTCAGATTTCTCACCAACAC-3′ |

### Example 4

### Expression and Purification of P450 3A4

E. coli XL-10 gold (Stratagene) was used as a host for expression cultures of P450 3A4. Starter cultures were grown overnight in LB media supplemented with 100 mg per litre ampicillin. 0.5 litre Terrific Broth media plus 100 mg per litre ampicillin and 1 mM thiamine and trace elements were inoculated with 1/100 dilution of the overnight starter cultures. The flasks were shaken at 37° C. until cell density OD 600 was 0.4 then δ-Aminolevulinic acid (ALA) was added to the cells at 0.5 mM for 20 min at 30° C. The cells were supplemented with 50 μM biotin then induced with optimum concentration of IPTG (30-100 μM) then shaken overnight at 30° C.

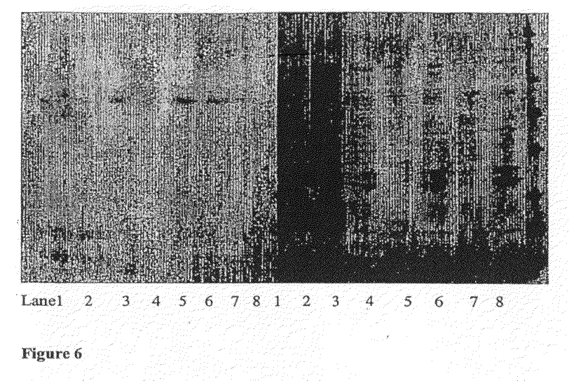

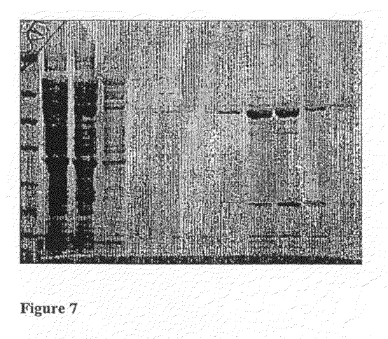

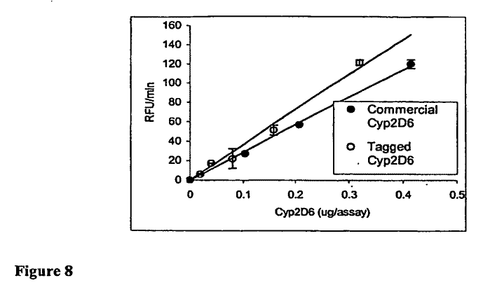

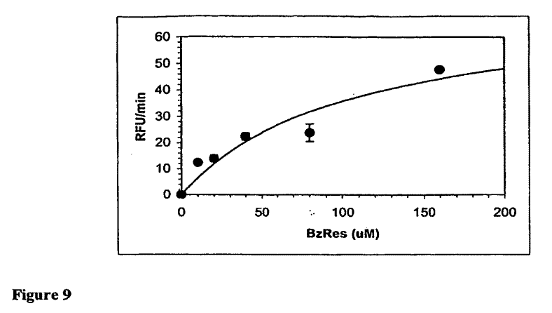

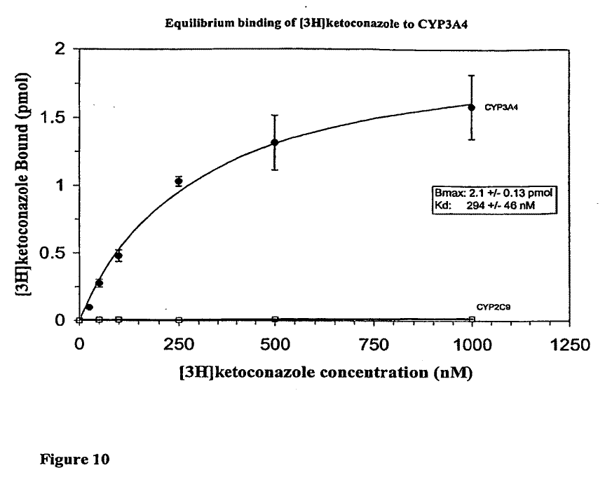

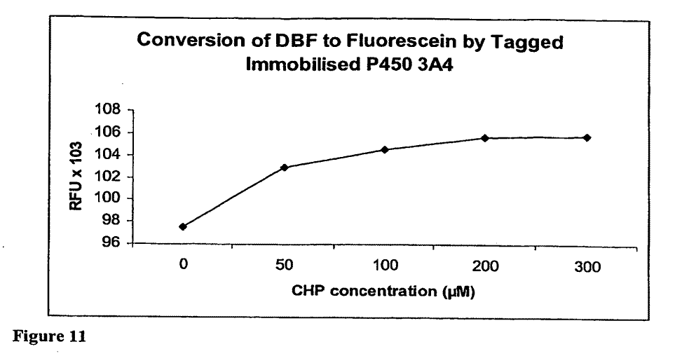

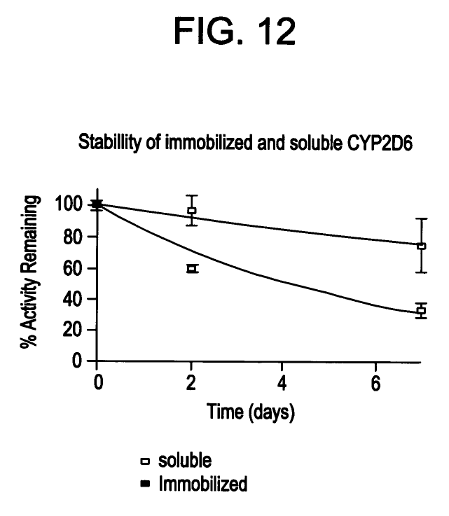

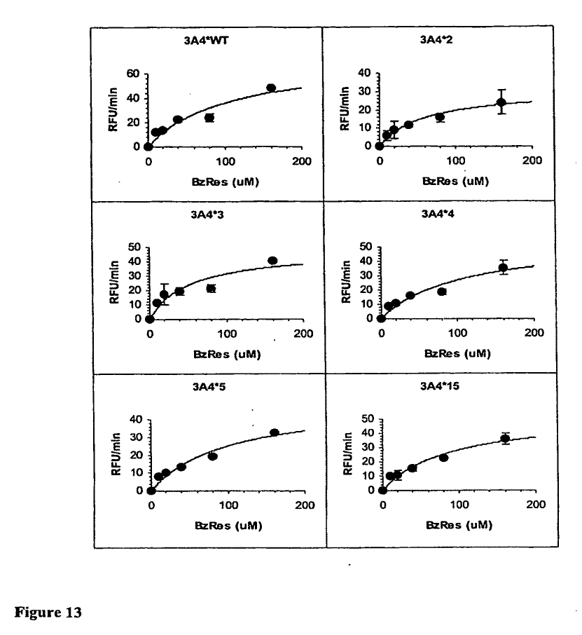

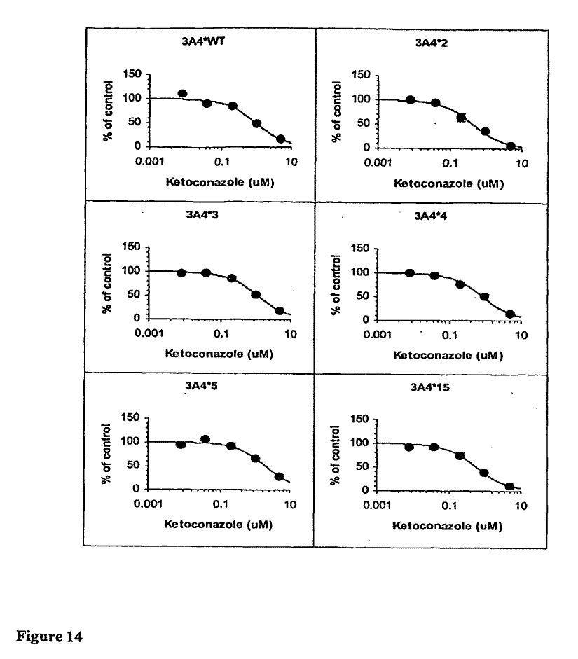

The E. coli cells from 0.5 litre cultures were divided into 50 ml aliquots, cells pelleted by centrifugation and cell pellets stored at −20° C. Cells from each pellet were lysed by resuspending in 5 ml buffer A (100 mM Tris buffer pH 8.0 containing 100 mM EDTA, 10 mM β-mercaptoethanol, 10× stock of Protease inhibitor cocktail—Roche 1836170, 0.2 mg/ml Lysozyme). After 15 minutes incubation on ice 40 ml of ice-cold deionised water was added to each resuspended cell pellet and mixed. 20 mM Magnesium Chloride and 5 μg/ml DNaseI were added. The cells were incubated for 30 min on ice with gentle shaking after which the lysed E. coli cells were pelletted by centrifugation for 30 min at 4000 rpm. The cell pellets were washed by resuspending in 10 ml buffer B (100 mM Tris buffer pH 8.0 containing 10 mM P-mercaptoethanol and a 10× stock of Protease inhibitor cocktail—Roche 1836170) followed by centrifugation at 4000 rpm. Membrane associated protein was then solubilised by the addition of 2 ml buffer C (50 mM potassium phosphate pH 7.4, 10× stock of Protease inhibitor cocktail-Roche 1836170, 10 mM β-mercaptoethanol, 0.5 M NaCl and 0.3% (v/v) Igepal CA-630) and incubating on ice with gentle agitation for 30 minutes before centrifugation at 10,000 g for 15 min at 4° C. and the supernatant (FIG. 6) was then applied to Talon resin (Clontech).

A 0.5 ml column of Ni-NTA agarose (Qiagen) was poured in disposable gravity columns and equilibrated with 5 column volumes of buffer C. Supernatant was applied to the column after which the column was successively washed with 4 column volumes of buffer C, 4 column volumes of buffer D (50 mM potassium phosphate pH 7.4, 10× stock of Protease inhibitor cocktail-Roche 1836170, 10 mM β-mercaptoethanol, 0.5 M NaCl and 20% (v/v) Glycerol) and 4 column volumes of buffer D+50 mM Imidazole before elution in 4 column volumes of buffer D+200 mM Imidazole (FIG. 7). 0.5 ml fractions were collected and protein containing fractions were pooled aliquoted and stored at −80° C.

### Example 5

### Determination of Heme Incorporation into P450s

Purified P450s were diluted to a concentration of 0.2 mg/ml in 20 mM potassium phosphate (pH 7.4) in the presence and absence of 10 mM KCN and an absorbance scan measured from 600-260 nm. The percentage bound heme was calculated based on an extinction coefficient ε₄₂₀ of 100 nM⁻¹ cm⁻¹.

### Example 6

### Reconstitution and Assay of Cytochrome P450 Enzymes into Liposomes with NADPH-Cytochrome P450 Reductase

Liposomes are prepared by dissolving a 1:1:1 mixture of 1,2-dilauroyl-sn-glycero-3-phosphocholine, 1,2-dileoyl-sn-glycero-3-phosphocholine, 1,2-dilauroyl-sn-glycero-3-phosphoserine in chloroform, evaporating to dryness and subsequently resuspending in 20 mM potassium phosphate pH 7.4 at 10 mg/ml. 4 μg of liposomes are added to a mixture of purified P450 2D6 (20 pmol), NADPH P450 reductase (40 pmol), cytochrome b5 (20 pmol) in a total volume of 10 μl and preincubated for 10 minutes at 37° C.

After reconstitution of cytochrome P450 enzymes into liposomes, the liposomes are diluted to 100 μl in assay buffer in a black 96 well plate, containing HEPES/KOH (pH 7.4, 50 mM), NADP+ (2.6 mM), glucose-6-phosphate (6.6 mM), MgCl₂ (6.6 mM) and glucose-6-phosphate dehydrogenase (0.4 units/ml). Assay buffer also contains an appropriate fluorogenic substrate for the cytochrome P450 isoform to be assayed: for P450 2D6 AMMC, for P450 3A4 dibenzyl fluorescein (DBF) or resorufin benzyl ether (BzRes) can be used and for 2C9 dibenzyl fluorescein (DBF). The reactions are stopped by the addition of ‘stopping solution’ (80% acetonitrile buffered with Tris) and products are read using the appropriate wavelength filter sets in a fluorescent plate reader (FIG. 8).

P450s can also be activated chemically by, for example, the addition of 200 μM cumene hydroperoxide in place of the both the co-enzymes and regeneration solution (FIG. 9).

In addition fluorescently measured rates of turnover can be measured in the presence of inhibitors.

### Example 7

### Detection of Drug Binding to Immobilised P450s CYP3A4

Purified CYP3A4 (10 μg/ml in 50 mM HEPES/0.01% CHAPS, pH 7.4) was placed in streptavidin immobiliser plates (Exiqon) (100 μl per well) and shaken on ice for 1 hour. The wells were aspirated and washed twice with 50 mM HEPES/0.01% CHAPS. [³H]-ketoconazole binding to immobilised protein was determined directly by scintillation counting. Saturation experiments were performed using [³H]ketoconazole (SCi/mmol, American Radiochemicals Inc., St. Louis) in 50 mM HEPES pH 7.4, 0.01% CHAPS and 10% Superblock (Pierce) (FIG. 1). Six concentrations of ligand were used in the binding assay (25-1000 nM) in a final assay volume of 100 μl. Specific binding was defined as that displaced by 100 μM ketoconazole. Each measurement was made in duplicate. After incubation for 1 hour at room temperature, the contents of the wells were aspirated and the wells washed three times with 150 μl ice cold assay buffer. 100 μl MicroScint 20 (Packard) was added to each well and the plates counted in a Packard TopCount microplate scintillation counter (FIG. 10).

### Example 8

### Chemical Activation of Tagged, Immobilised CYP3A4

CYP3A4 was immobilised in streptavidin immobiliser plates as described in Example 7 and was then incubated with dibenzyl fluorescein and varying concentrations (0-300 μM) of cumene hydrogen peroxide. End point assays demonstrated that the tagged, immobilised CYP3A4 was functional in a turn-over assay with chemical activation (FIG. 11).

### Example 9

### Immobilisation of P450s through Gel Encapsulation of Liposomes or Microsomes

After reconstitution of cytochrome P450 enzymes together with NADPH-cytochrome P450 reductase in liposomes or microsomes, these can then be immobilised on to a surface by encapsulation within a gel matrix such as agarose, polyurethane or polyacrylamide.

For example, low melting temperature (LMT) (1% w/v) agarose was dissolved in 200 mM potassium phosphate pH 7.4. This was then cooled to 37° C. on a heating block. Microsomes containing cytochrome P450 3A4, cytochrome b5 and NADPH-cytochrome P450 reductase were then diluted into the LMT agarose such that 50 μl of agarose contained 20, 40 and 20 μmol of P450 3A4, NADPH-cytochrome P450 reductase and cytochrome b5 respectively. 50 μl of agarose-microsomes was then added to each well of a black 96 well microtitre plate and allowed to solidify at room temperature.

To each well, 100 μl of assay buffer was added and the assay was conducted as described previously (for example, Example 6) for conventional reconstitution assay. From the data generated a comparison of the fundamental kinetics of BzRes oxidation and ketoconazole inhibition was made (Table 5) which showed that the activity of the CYP3A4 was retained after gel-encapsulation.

TABLE 5
Comparison of kinetic parameters for Bz Rez oxidation and inhibition
by ketoconazole for cytochrome P450 3A4 microsomes in solution
and encapsulated in agarose¹.

| | Gel encapsulated | Soluble |
|---|---|---|
| BzRes Oxidation | | |
| K_{M} (μM) | 49 (18) | 20 (5) |
| Vₘₐₓ (% of soluble) | 50 (6) | 100 (6) |
| Ketoconazole inhibition | | |
| IC50 (nM) | 86 (12) | 207 (54) |

¹For estimation of K_{M} and Vₘₐₓ for BzRes assays were performed in the presence of varying concentrations of BzRes up to 320 μM. Ketoconazole inhibition was performed at 50 μM BzRes with 7 three-fold dilutions of ketoconazole from 5 μM.
Values in parenthesis indicate standard errors derived from the curve fitting.

The activity of the immobilised P450s was assessed over a period of 7 days (FIG. 12). Aliquots of the same protein preparation stored under identical conditions, except that they were not gel-encapsulated, were also assayed over the same period, which revealed that the gel encapsualtion confers significant stability to the P450 activity.

### Example 10

### Quantitative Determination of Affect of 3A4 Polymorphisms on Activity

Purified cytochrome P450 3A4 isoforms *1, *2, *3, *4, *5 & *15 (approx 1 μg) were incubated in the presence of BzRes and cumene hydrogen peroxide (200 μM) in the absence and presence of ketoconazole at room temperature in 200 mM KPO₄ buffer pH 7.4 in a total volume of 100 μl in a 96 well black microtitre plate. A minimum of duplicates were performed for each concentration of BzRes or ketoconazole. Resorufin formation of was measured over time by the increase in fluorescence (520 nm and 580 nm excitation and emission filters respectively) and initial rates were calculated from progress curves (FIG. 13).

For estimation of K_{M}^{app} and Vₘₐₓ^{app} for BzRes, background rates were first subtracted from the initial rates and then were plotted against BzRes concentration and curves were fitted describing conventional Michaelis-Menton kinetics:

V=Vₘₐₓ/(1+(K_{M}/S))

where V and S are initial rate and substrate concentration respectively. Vₘₐₓ values were then normalized for cytochrome P450 concentration and scaled to the wild-type enzyme (Table 6).

For estimation of IC₅₀ for ketoconazole, background rates were first subtracted from the initial rates which were then converted to a % of the uninhibited rate and plotted against ketoconazole concentration (FIG. 14). IC₅₀ inhibition curves were fitted using the equation:

V=100/(1+(I/IC₅₀))

where V and I are initial rate and inhibitor concentration respectively. The data obtained is shown in Table 6:

TABLE 6
Kinetic parameters for BzRes turnover and its inhibition by
ketoconazole for cytochrome P450 3A4 isoforms.

| | Vₘₐₓ BzRes | K_{M} BzRes (μM) | IC₅₀ ketoconazole (μM) |
|---|---|---|---|
| 3A4*WT | 100 (34) | 104 (25) | 0.91 (0.45) |
| 3A4*2 | 65 (9) | 62 (4) | 0.44 (0.11) |
| 3A4*3 | 93 (24) | 54 (13) | 1.13 (0.16) |
| 3A4*4 | 69 (22) | 111 (18) | 0.88 (0.22) |
| 3A4*5 | 59 (16) | 101 (11) | 1.96 (0.96) |
| 3A4*15 | 111 (23) | 89 (11) | 0.59 (0.20) |

The parameters were obtained from the fits of Michaelis-Menton and IC₅₀ inhibition curves to the data in FIGS. 13 & 14. Values in parenthesis are standard errors obtained from the curve fits.

### Example 11

### Array-Based Assay of Immobilised CYP3A4 Polymorphisms

Cytochrome P450 polymorphisms can be assayed in parallel using an array format to identify subtle differences in activity with specific small molecules. For example, purified cytochrome P450 3A4 isoforms *1, *2, *3, *4, *5 & *15 can be individually reconstituted in to liposomes with NADPH-cytochrome P450 reductase as described in Example 9. The resultant liposomes preparation can then be diluted into LMP agarose and immobilised into individual wells of a black 96 well microtitre plate as described in Example 9. The immobilised proteins can then be assayed as described in Example 9 by adding 100 μl of assay buffer containing BzRes +/− ketoconazole to each well.

Chemical activation (as described in Example 10) can also be used in an array format. For example, purified cytochrome P450 3A4 isoforms *1, *2, *3, *4, *5 & *15 can be individually reconstituted in to liposomes without NADPH-cytochrome P450 reductase and the resultant liposomes can be immobilised via encapsulation in agarose as described in Example 9. The cytochrome P450 activity in each well can then be measured as described in Example 10 by 100 μl of 200 mM KPO₄ buffer pH 7.4 containing BzRes and cumene hydrogen peroxide (200 μM), +/− ketoconazole, to each well.

### Example 12

### Array-Based Assay of a Panel of Wild-Type Cytochrome P450s

Baculovirally-expressed H. sapiens cytochrome P450s CYP1A2, CYP2A6, CYP2B6, CYP2C8, CYP2C9*1, CYP2C9*2, CYP2C9*3, CYP2C19, CYP2D6, CYP2 μl, CYP3A4, and CYP3A5 (Sigma) can be reconstituted in to microsomes (Sigma) with NADPH-cytochrome P450 reductase and immobilised via gel encapsulation as described in Example 9. Activity assays can then carried out in parallel on the array of immobilised P450s as described in Example 9 using appropriate fluorescent substrates for each P450. The interaction of the arrayed P450s with, for example, the drug cyclosporin A can then be determined by measuring the extent to which the turn-over of the relevant fluorescent substrate by any one P450 is modulated by the presence of the drug, as described in Example 10. Alternatively, the formation of metabolites can be measured using LC-MS methods since these are typically compatible with loading samples a 96-well format.

### Example 13

### Array-Based Comparison of Rat and Human Cytochrome P450 Activity

H. sapiens CYP2C9, CYP2D6, CYP3A4 and Rattus norvegicus CYP2C9, CYP2D6, CYP3A4 are cloned into vector pBJW102.2 and the recombinant proteins are then IQ expressed and purified according to the protocols described in Example 4. The purified recombinant proteins can then incorporated into liposomes with NADPH-cytochrome P450 reductase and immobilised via gel encapsulation as described in Example 9. Activity assays can be carried out in parallel on the array of immobilised P450s as, for example, described in Example 9.

### Example 14

### A phase 1 and Phase 2 Co-Array

Co-arrays of phase 1 and phase 2 enzymes are created by, for example, reconstituting twelve liposome preparations containing NADPH-cytochrome P450 reductase together with, individually, the cytochrome P450s CYP1A2, CYP2A6, CYP2B6, CYP2C8, CYP2C9*1, CYP2C9*2, CYP2C9*3, CYP2C19, CYP2D6, CYP2E1, CYP3A4, and CYP3A5. These 12 liposome preparations are then each immobilised via agarose gel encapsulation in to 12 separate wells of a 96-well microtitre plate. To each well is then added a solution containing the human phase 2 enzyme glutathione S-transferase P1. The test compound, for example paracetamol is then applied to each well and the formation and identity of conjugated metabolites can be detected by LC-MC methods.

### REFERENCES

1. Johansson, I., Oscarson, M., Yue, Q. Y., Bertilsson, L., Sjoqvist, F. & Ingelman-Sundberg, M. (1994) Mol Pharmacol 46, 452-9.
2. Sachse, C., Brockmoller, J., Bauer, S. & Roots, I. (1997) Am J Hum Genet. 60, 284-95.
3. Griese, E. U., Zanger, U. M., Brudermams, U., Gaedigk, A., Mikus, G., Morike, K., Stuven, T. & Eichelbaum, M. (1998) Pharmacogenetics 8, 15-26.
4. Marez, D., Legrand, M., Sabbagh, N., Guidice, J. M., Spire, C., Lafitte, J. J., Meyer, U. A. & Broly, F. (1997) Pharmacogenetics 7, 193-202.
5. Aklillu, E., Persson, I., Bertilsson, L., Johansson, I., Rodrigues, F. & Ingelman-Sundberg, M. (1996) J Pharmacol Exp Ther 278, 441-6.
6. Dandara, C., Masimirembwa, C. M., Magimba, A., Sayi, J., Kaaya, S., Sommers, D. K., Snyman, J. R. & Hasler, J. A. (2001) Eur J Clin Pharmacol 57, 11-7.
7. Aithal, G. P., Day, C. P., Kesteven, P. J. & Daly, A. K. (1999) Lancet 353, 717-9.
8. Imai, J., Ieiri, I., Mamiya, K, Miyahara, S., Furuumi, H., Nanba, E., Yamane, M., Fukumaki, Y., Ninomiya, H., Tashiro, N., Otsubo, K. & Higuchi, S. (2000) Pharmacogenetics 10, 85-9.
9. Dickmann, L. J., Rettie, A. E., Kneller, M. B., Kim, R. B., Wood, A. J., Stein, C. M., Wilkinson, G. R. & Schwarz, U. I. (2001) Mol Pharmacol 60, 382-7.
10. Sata, F., Sapone, A., Elizondo, G., Stocker, P., Miller, V. P., Zheng, W., Raunio, H., Crespi, C. L. & Gonzalez, F. J. (2000) Clin Pharmacol Ther 67, 48-56.
11. Eiselt, R., Domanski, T. L., Zibat, A., Mueller, R., Presecan-Siedel, E., Hustert, E., Zanger, U. M., Brockinoller, J., KIenk, H. P., Meyer, U. A., Khan, K. K., He, Y. A., Halpert, J. R. & Wojnowski, L. (2001) Pharmacogenetics 11, 447-58.
12. Dai, D., Tang, J., Rose, R., Hodgson, E., Bienstock, R. J., Mohrenweiser, H. W. & Goldstein, J. A. (2001) J Pharmacol Exp Ther 299, 825-31.
13. Hsieh, K. P., Lin, Y. Y., Cheng, C. L., Lai, M. L., Lin, M. S., Siest, J. P. & Huang, J. D. (2001) Drug Metab Dispos 29, 268-73.

1-36. (canceled)

37. A protein array comprising a surface having a plurality of spatially defined locations wherein at each location there are deposited at least two protein moieties which are capable of forming a complex characterized in that said complex is transiently formed.

38. The protein array of claim 37 wherein the complex is transiently formed during catalysis.

39. A protein array comprising a surface having a plurality of spatially defined locations wherein at each location there are deposited at least two protein moieties characterized in that said protein moieties at each location act sequentially on a substrate of interest.

40. The protein array of claim 37 wherein at least one of said protein moieties at each location is capable of being membrane-associated or membrane-bound or has been modified to interact with a non-polar or amphipathic molecule.

41. The protein array of claim 37 wherein at least one of said moieties at each location is a drug metabolizing enzyme.

42. A protein array comprising a surface upon which are deposited at spatially defined locations at least two protein moieties characterized in that said protein moieties are derived from one or more drug metabolizing enzymes.

43. The protein array of claim 41 wherein at least one of said protein moieties at each location is a P450 protein.

44. The protein array of claim 39 wherein said protein moieties are attached to said surface through a marker moiety appended to each protein moiety.

45. The protein array of claim 37 wherein said protein moieties are incorporated into a membrane, vesicle or liposome which is immobilised in proximity to said surface.

46. The protein array of claim 41 wherein said drug metabolizing enzymes are selected from the group consisting of cytochrome P450s, flavin monooxygenases, UDP glycosyltransferases, glutathione S-transferases, sulfotransferases and N-acetyltransferases.

47. The protein array of claim 39 said array comprises one or more Phase 1 drug metabolizing enzymes and one or more Phase 2 drug metabolizing enzymes.

48. The protein array of claim 41 wherein said drug metabolizing enzymes are H. sapiens cytochrome P450s and are selected from the group consisting of COP1A2, CYP2A6, CYP2B6, CYP2C8, CYP2C9*1, CYP2C9*2, CYP2C9*3, CYP2C19, CYP2D6, CYP2E1, CYP3A4 and CYP3A5.

49. The protein array of claim 41 wherein one or more of said drug metabolizing enzymes are derived from different ethnic groups, different genders, different mammalian species, or different mutant versions of a wild type enzyme.

50. A method of making a protein array comprising the steps of:
a) providing two or more drug metabolizing enzymes of interest from a recombinant source, a native source or a synthetic source; and
b) depositing said proteins at spatially defined locations on a surface to make said protein array.

51. The method of claim 50, wherein said drug metabolizing enzymes are brought into contact with the array in admixture with other protein molecules and deposition on the array occurs with simultaneous purification of the protein moiety on the array via a tag incorporated in the protein moiety.

52. The method of claim 50, wherein said drug metabolizing enzymes are deposited with other proteins from an expression host cell on a surface at spatially defined locations to make said protein array.

53. An array made by the method of claim 50.

54. A method of making a protein array comprising the steps of:
a) providing one or more proteins from either recombinant, native or synthetic sources incorporated in purified or partially purified membrane or membrane-like preparations; and
b) arraying said proteins by encapsulation of said membrane or membrane-like preparations into a gel matrix which is deposited on the surface.

55. A method of making an array of drug metabolizing enzymes comprising the steps of:
a) providing drug metabolizing enzymes from either recombinant, native or synthetic sources in the form of purified or partially purified membrane or membrane-like preparations; and
b) arraying said drug metabolizing enzymes either by deposition of said membrane or membrane-like preparations onto a suitable surface capable of capturing the membranes or by encapsulation of said membrane or membrane-like preparations into a gel matrix which is deposited on the surface.

56. The method of claim 55 wherein one or more of said membrane or membrane-like preparations contains two or more different proteins.

57. The method of claim 56 wherein said two or more different proteins are capable of forming a complex with each other.

58. The method of claim 57 wherein said complex is transiently formed.

59. The method of claim 56 wherein said two or more different proteins act sequentially on a substrate of interest.

60. A method of screening a set of protein moieties for molecules which interact with one or more proteins comprising the steps of:
a) bringing one or more test molecules into contact with an array as claimed in claim 37 which carries said set of protein moieties; and
b) detecting an interaction between one or more test molecules and one or more proteins on the array.

61. A method of simultaneously determining the relative properties of members of a set of protein moieties, comprising the steps of:
a) bringing an array as claimed in claim 37 which carries said set of protein moieties into contact with one or more test substances, and
b) observing the interaction of said test substances with the set members on the array.

62. The method of claim 61 wherein one or more of said protein moieties are drug metabolizing enzymes and wherein said enzymes are activated by contact with an accessory protein or by chemical treatment.

63. A method for determining a gender differences in drug metabolism comprising the steps of:
a) providing a male drug metabolizing enzyme array of claim 41;
b) providing a female drug metabolizing enzyme array of claim 41;
c) bringing said arrays into contact with one or more test substances; and
d) observing a difference in the interaction of said test substances with the drug metabolizing enzymes of the two array.

64. A method for determining an ethnic difference in drug metabolism comprising the steps of:
a) providing drug metabolizing enzyme array of claim 41 from a first ethnic group;
b) providing drug metabolizing enzyme array of claim 41 from a second ethnic group;
c) bringing said arrays into contact with one or more test substances; and
d) observing a difference in the interaction of said test substances with the drug metabolizing enzymes of the two array.

65. A method for determining a difference in drug metabolism in two mammalian species comprising the steps of:
a) providing drug metabolizing enzyme array of claim 41 from a first mammalian species;
b) providing drug metabolizing enzyme array of claim 41 from a second mammalian species;
c) bringing said arrays into contact with one or more test substances; and
d) observing a difference in the interaction of said test substances with the drug metabolizing enzymes of the two array.

66. The method of claim 29 wherein said mammalian species are human and rat.

67. A method for determining the cytotoxicity of drug metabolites comprising the steps of:
a) providing drug metabolizing enzyme array of claim 41;
b) overlaying said array with cells that act as reporters in a cytotoxicity assay;
c) bringing said arrays into contact with one or more test substances; and
d) observing an interaction between any metabolites produced by the drug metabolizing enzyme and said cells.

68. A method for determining and quantitating of metabolic pathways for small molecules comprising the steps of:
a) providing drug metabolizing enzyme array of claim 41;
b) bringing said arrays into contact with one or more small molecules; and
c) observing in interaction between said one or more small molecules and said drug metabolizing enzyme array to determine and quantitate said metabolic pathway.

69. A method for screening compounds for their ability to bind and inhibit individual drug metabolizing enzymes comprising the steps of:
a) providing drug metabolizing enzyme array of claim 41;
b) bringing said arrays into contact with one or more compounds; and
c) observing the binding or inhibition of said compounds with said drug metabolizing enzyme array.

70. A method for determining the induction of P450 expression by one or more drugs comprising the steps of:
a) providing a p450 array of claim 46 from a normal tissue;
b) providing a P450 array of claim 46 from a tissue treated with said one or more drugs; and
c) observing a difference in the distribution of said P450 array to determine an induction of P450 expression by said one or more drugs.

71. A method for analyzing the effects of mutation on the activity of a drug metabolizing enzyme of interest comprising the steps of:
a) providing a first drug metabolizing enzyme array of claim 41wherein said drug metabolizing enzyme is mutated;
b) providing a second drug metabolizing enzyme array of claim 41;
c) bringing said arrays into contact with one or more compounds; and
d) observing a difference in interaction between said one or more compounds and said arrays.

72. A method of expressing and purifying a drug metabolizing enzyme, comprising the steps of:
a) expressing a drug metabolizing enzyme of interest in a host cell;
b) subjecting said host cell to conditions suitable to lyse the cell;
c) obtaining a membrane associated cell fraction from the lysed cell:
d) solubilizing said membrane associated cell fraction by the addition of a detergent;
e) after an incubation period sufficient to solubilize the drug metabolizing enzyme protein contained in said membrane associated cell fraction, performing a further centrifugation step to produce a supernatant containing said drug metabolizing enzyme protein; and
f) subjecting said supernatant to chromatography to purify said drug metabolizing enzyme protein.
